SEQUENCE LISTING (11 sequences)

SEQ ID NO: 1 - protein, 14 aa, synthetic construct
TGSSSNIGAGYDVH

SEQ ID NO: 2 - protein, 7 aa, synthetic construct
GDSNRPS

SEQ ID NO: 3 - protein, 11 aa, synthetic construct
QSFDNSLSAYV

SEQ ID NO: 4 - protein, 111 aa, synthetic construct
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVL

SEQ ID NO: 5 - protein, 217 aa, synthetic construct
ESVLTQPPSVSGAPGQRVTISCTGSSSNIGAGYDVHWYQQLPGTAPKLLIYGDSNRPSGVPDRFSVSKSGASASLAITGLQAEDEADYYCQSFDNSLSAYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 5 aa, synthetic construct
GYYWS

SEQ ID NO: 7 - protein, 16 aa, synthetic construct
EINHAGSTNYNPSLKS

SEQ ID NO: 8 - protein, 13 aa, synthetic construct
LPTRWVTTRYFDL

SEQ ID NO: 9 - protein, 121 aa, synthetic construct
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSS

SEQ ID NO: 10 - protein, 448 aa, synthetic construct
EVQLQQWGAGLLKPSETLSLTCAVYGGSFSGYYWSWIRQPPGKGLEWIGEINHAGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 5 aa, synthetic construct
  SITE 1: note = N,N-dimethyl-valine
  SITE 3: note = N-methyl-valine
  SITE 5: note = proline-tert-butylamide
VVVPP

(19) United States
(12) Patent Application Publication
(10) Pub. No.: US 2025/0011453 A1
(43) Pub. Date: Jan. 9, 2025

(54) METHODS FOR TREATING CANCER USING A COMBINATION OF A PD-1 ANTAGONIST, AN ILT4 ANTAGONIST, AND CHEMOTHERAPEUTIC AGENTS

(71) Applicant: Merck Sharp & Dohme LLC, Rahway, NJ (US)

(72) Inventor: Rachel A. Altura, Westerly, RI (US)

(73) Assignee: Merck Sharp & Dohme LLC, Rahway, NJ (US)

(21) Appl. No.: 18/580,255

(22) PCT Filed: Jul. 18, 2022
(86) PCT No.: PCT/US2022/037414

§ 371 Date: Jan. 18, 2024

Related U.S. Application Data
(60) Provisional application No. 63/223,739, filed on Jul. 20, 2021.

(51) Int. Cl. C07K 16/28; A61K 31/337; A61K 31/519; A61K 33/243; A61P 35/00

(52) U.S. Cl. CPC C07K 16/2896; A61K 31/337; A61K 31/519; A61K 33/243; A61P 35/00; C07K 16/2803; C07K 2317/24; C07K 2317/51; C07K 2317/515; C07K 2317/565

(57) ABSTRACT

Provided herein are methods of treating cancer (e.g., ovarian, mesothelioma, triple-negative breast cancer), which comprise administering to a human patient in need thereof: (a) a PD-1 antagonist; (b) an ILT4 antagonist; and (c) one or more chemotherapeutic agents. Also provided are pharmaceutical compositions and kits containing such agents for the treatment of cancer.

### I. FIELD

Provided herein are methods for treating cancer (e.g., mesothelioma, ovarian cancer, or triple-negative breast cancer (TNBC)) using a combination of (a) a programmed death 1 protein (PD-1) antagonist, (b) an immunoglobulin-like transcript 4 (ILT4) antagonist, and (c) one or more chemotherapeutic agents.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Jul. 8, 2022, is named 25271-WO-PCT_SL.XML and is 13 KB in size.

### II. BACKGROUND OF THE INVENTION

PD-1 is recognized as an important player in immune regulation and the maintenance of peripheral tolerance. Immune checkpoint therapies targeting PD-1 or its ligand (e.g., PD-L1) have resulted in groundbreaking improvements in clinical response in multiple human cancer types (Brahmer et al., N Engl J Med, 366:2455-2465 (2012); Garon et al., N Engl J Med, 372:2018-2028 (2015); Hamid et al., N Engl J Med, 369:134-144 (2013); Robert et al., Lancet, 384:1109-1117 (2014); Robert et al., N Engl J Med, 372:2521-2532 (2015); Robert et al., N Engl J Med, 372:320-330 (2015); Topalian et al., N Engl J Med, 366:2443-2454 (2012); Topalian et al., J Clin Oncol, 32:1020-1030 (2014); Wolchok et al., N Engl J Med, 369:122-133 (2013)). Immune therapies targeting the PD-1 axis include monoclonal antibodies directed to the PD-1 receptor (e.g., KEYTRUDAR (pembrolizumab), Merck and Co., Inc., Kenilworth, NJ; OPDIVOR (nivolumab), Bristol-Myers Squibb Company, Princeton, NJ) and those that bind to the PD-L1 ligand (e.g., TECENTRIQR (atezolizumab), Genentech, San Francisco, CA).

Another common strategy used by tumor cells to escape innate and adaptive immune response is associated with aberrant expression of human leukocyte antigen (HLA)-G (Curigliano et al. Clin Cancer Res. 2013 and Gonzalez et al. Crit Rev Clin Lab Sci. 2012). HLA-G can directly inhibit immune cell function through receptor binding and/or through trogocytosis and impairment of chemotaxis (Morandi et al. Cytokine Growth Factor Review. 2014 and Lin et al. Mol Med. 2015). Antibody-mediated blockade of HLA-G function in transgenic mouse models has been shown to inhibit tumor development and block expansion of myeloid-derived suppressor cells (MDSC) (Loumange et al. Int J Cancer. 2014, Lin et al. Hum Immunol. 2013, and Agaugue et al. Blood. 2011). HLA-G binding to ILT4 can directly inhibit the function of monocytes, dendritic cells, and neutrophils, thus impairing the innate immune anti-tumor response. Accordingly, ILT4 blockade was predicted to relieve suppression of tolerogenic myeloid cells in the tumor microenvironment, and this has been supported by experimental evidence (Chen et al., J. Clin. Invest. 2018, 128 (12): 5647-5662).

It has been proposed that the efficacy of anti-PD-1 or anti-PD-L1 antagonistic antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents. However, there are no clear guidelines as to which agent combined with the anti-PD-1 or anti-PD-L1 antibodies may be effective or in which cancer types the combination may enhance the efficacy of treatment. Thus, there is an unmet need in the art for high efficacy therapeutic combinations that can generate a robust immune response to cancer.

### III. SUMMARY

The present disclosure provides methods of treating cancer (e.g., mesothelioma, ovarian cancer, or TNBC) using a combination of a PD-1 antagonist, an ILT4 antagonist, and one or more chemotherapeutic agents.

The present disclosure further provides kits including a PD-1 antagonist, an ILT4 antagonist, and one or more chemotherapeutic agents.

Also provided herein are uses of a therapeutic combination for treating cancer (e.g., mesothelioma, ovarian cancer, or TNBC), and the therapeutic combination includes a PD-1 antagonist, an ILT4 antagonist, and one or more chemotherapeutic agents.

In one aspect, provided herein is a method of treating cancer, comprising administering to a human patient in need thereof:
(a) a PD-1 antagonist;
(b) an ILT4 antagonist;
(c) cisplatin; and
(d) pemetrexed.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., NSCLC), pleural mesothelioma, gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In some embodiments, the cancer is relapsed. In other embodiments, the cancer is refractory. In yet other embodiments, the cancer is relapsed and refractory.

In one embodiment, the cancer is mesothelioma. In another embodiment, the mesothelioma is advanced mesothelioma. In yet another embodiment, the mesothelioma is recurrent mesothelioma.

In another aspect, provided herein is a kit comprising:
(a) a PD-1 antagonist;
(b) an ILT4 antagonist;
(c) cisplatin; and
(d) pemetrexed.

In certain embodiments, the kit further comprises instructions for administering to a human patient the PD-1 antagonist, the ILT4 antagonist, cisplatin, and pemetrexed.

In still another aspect, provided herein is use of a therapeutic combination for treating cancer in a human patient, wherein the therapeutic combination comprises:
(a) a PD-1 antagonist;
(b) an ILT4 antagonist;
(c) cisplatin; and
(d) pemetrexed.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., NSCLC), pleural mesothelioma, gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In some embodiments, the cancer is relapsed. In other embodiments, the cancer is refractory. In yet other embodiments, the cancer is relapsed and refractory.

In one embodiment, the cancer is mesothelioma. In another embodiment, the mesothelioma is advanced mesothelioma. In yet another embodiment, the mesothelioma is recurrent mesothelioma.

In another aspect, provided herein is a method of treating cancer, comprising administering to a human patient in need thereof:
(a) a PD-1 antagonist;
(b) an ILT4 antagonist; and
(c) a mitosis inhibitor.

In some embodiments, the mitosis inhibitor is paclitaxel. In other embodiments, the mitosis inhibitor is docetaxel.

In certain embodiments of such a method, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer (NSCLC), pleural mesothelioma, gastric cancer, colorectal cancer, cervical cancer, synovial sarcoma, head and neck cancer, squamous cell carcinoma, lymphoma, diffuse large B-cell lymphoma, non-Hodgkin lymphoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In one embodiment, the cancer is breast cancer. In another embodiment, the breast cancer is TNBC. In yet another embodiment, the cancer is ovarian cancer.

In another aspect, provided herein is a kit comprising:
(a) a PD-1 antagonist;
(b) an ILT4 antagonist; and
(c) a mitosis inhibitor.

In some embodiments, the mitosis inhibitor is paclitaxel. In other embodiments, the mitosis inhibitor is docetaxel.

In certain embodiments, the kit further comprises instructions for administering to a human patient the PD-1 antagonist, the ILT4 antagonist, and paclitaxel.

In some embodiments, the kit further comprises instructions for administering to a human patient the PD-1 antagonist, the ILT4 antagonist, and docetaxel.

In yet another aspect, provided herein is use of a therapeutic combination for treating cancer in a human patient, wherein the therapeutic combination comprises:
(a) a PD-1 antagonist;
(b) an ILT4 antagonist; and
(c) a mitosis inhibitor.

In some embodiments, the mitosis inhibitor is paclitaxel. In other embodiments, the mitosis inhibitor is docetaxel.

In various embodiments of such use, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer, pleural mesothelioma, gastric cancer, colorectal cancer, cervical cancer, synovial sarcoma, head and neck cancer, squamous cell carcinoma, lymphoma, diffuse large B-cell lymphoma, non-Hodgkin lymphoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In one embodiment, the cancer is breast cancer. In another embodiment, the breast cancer is TNBC. In yet another embodiment, the cancer is ovarian cancer.

In certain embodiments of various methods, kits, or uses provided herein, the PD-1 antagonist is an anti-human PD-1 monoclonal antibody or antigen binding fragment thereof.

In certain embodiments of various methods, kits, or uses provided herein, the PD-1 antagonist is an anti-human PD-L1 monoclonal antibody or antigen binding fragment thereof.

In some embodiments of various methods, kits, or uses provided herein, the anti-human PD-1 monoclonal antibody is a humanized antibody.

In other embodiments of various methods, kits, or uses provided herein, the anti-human PD-1 monoclonal antibody is a human antibody.

In certain embodiments of various methods, kits, or uses provided herein, the ILT4 antagonist is an anti-human ILT4 monoclonal antibody or antigen binding fragment thereof.

In some embodiments of various methods, kits, or uses provided herein, the anti-human ILT4 monoclonal antibody is a humanized antibody.

In other embodiments of various methods, kits, or uses provided herein, the anti-human ILT4 monoclonal antibody is a human antibody.

In one embodiment of various methods, kits, or uses provided herein, the anti-human PD-1 monoclonal antibody is pembrolizumab.

In another embodiment of various methods, kits, or uses provided herein, the anti-human PD-1 monoclonal antibody is nivolumab.

In another embodiment of various methods, kits, or uses provided herein, the anti-human PD-1 monoclonal antibody is cemiplimab.

In certain embodiments of various methods, kits, or uses provided herein, the anti-human ILT4 monoclonal antibody comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively.

In some embodiments of various methods, kits, or uses provided herein, the anti-human ILT4 monoclonal antibody comprises a V_{L} region comprising an amino acid sequence as set forth in SEQ ID NO:4, and a V_{H} region comprising an amino acid sequence as set forth in SEQ ID NO:9.

In other embodiments of various methods, kits, or uses provided herein, the anti-human ILT4 monoclonal antibody comprises a light chain comprising or consisting of an amino acid sequence as set forth in SEQ ID NO:5 and a heavy chain comprising or consisting of an amino acid sequence as set forth in SEQ ID NO:10.

In one specific embodiment of various methods, kits, or uses provided herein, the PD-1 antagonist is pembrolizumab; and the ILT4 antagonist is a monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively.

In one specific embodiment of various methods, pharmaceutical compositions, kits, or uses provided herein, the PD-1 antagonist is nivolumab; and the ILT4 antagonist is a monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively.

In one specific embodiment of various methods, pharmaceutical compositions, kits, or uses provided herein, the PD-1 antagonist is cemiplimab; and the ILT4 antagonist is a monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively.

In some embodiments of various methods described herein, the anti-human PD-1 monoclonal antibody or antigen binding fragment thereof is pembrolizumab, the human patient is administered 200 mg, 240 mg, or 2 mg/kg pembrolizumab once every three weeks. In one embodiment, the human patient is administered 200 mg pembrolizumab once every three weeks. In one embodiment, the human patient is administered 240 mg pembrolizumab once every three weeks. In one embodiment, the human patient is administered 2 mg/kg pembrolizumab once every three weeks.

In certain embodiments of various methods described herein, the anti-human PD-1 monoclonal antibody or antigen binding fragment thereof is pembrolizumab, the human patient is administered 400 mg pembrolizumab once every six weeks.

In other embodiments of various methods described herein, the anti-human PD-1 monoclonal antibody or antigen binding fragment thereof is nivolumab, the human patient is administered 240 mg or 3 mg/kg nivolumab once every two weeks. In one specific embodiment, the human patient is administered 240 mg nivolumab once every two weeks. In one specific embodiment, the human patient is administered 3 mg/kg nivolumab once every two weeks.

In still other embodiments of various methods described herein, the anti-human PD-1 monoclonal antibody or antigen binding fragment thereof is nivolumab, the human patient is administered 480 mg nivolumab once every four weeks.

In yet other embodiments of various methods described herein, the anti-human PD-1 monoclonal antibody or antigen binding fragment thereof is cemiplimab, the human patient is administered 350 mg cemiplimab once every three weeks.

In still other embodiments of various methods described herein, the anti-human ILT4 monoclonal antibody or antigen binding fragment thereof comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively, the human patient is administered from about 100 mg to about 1600 mg of the anti-human ILT4 antibody once every three weeks. In some embodiments, the human patient is administered 100, 200, 300, 400, 800, 1000, or 1600 mg of the anti-human ILT4 antibody once every three weeks. In one specific embodiment, the human patient is administered 100 mg of the anti-human ILT4 antibody once every three weeks. In one specific embodiment, the human patient is administered 200 mg of the anti-human ILT4 antibody once every three weeks. In one specific embodiment, the human patient is administered 300 mg of the anti-human ILT4 antibody once every three weeks. In one specific embodiment, the human patient is administered 400 mg of the anti-human ILT4 antibody once every three weeks. In one specific embodiment, the human patient is administered 800 mg of the anti-human ILT4 antibody once every three weeks. In one specific embodiment, the human patient is administered 1000 mg of the anti-human ILT4 antibody once every three weeks. In one specific embodiment, the human patient is administered 1600 mg of the anti-human ILT4 antibody once every three weeks.

In certain embodiments of various methods described herein, the human patient is administered cisplatin at from about 20 mg/m² to about 100 mg/m² and pemetrexed at from about 200 mg/m² to about 750 mg/m², and cisplatin and pemetrexed are administered once every three weeks.

In some embodiments of various methods described herein, the human patient is administered cisplatin at 20 mg/m², 25 mg/m², 50 mg/m², 75 mg/m², 100 mg/m² and pemetrexed at 250 mg/m², 375 mg/m², or 500 mg/m², and cisplatin and pemetrexed are administered once every three weeks.

In other embodiments of various methods described herein, the human patient is administered 75 mg/m² cisplatin and 500 mg/m² pemetrexed, and cisplatin and pemetrexed are administered once every three weeks.

Thus, in some embodiments, the human patient is administered:
(a) 200 mg, 240 mg, or 2 mg/kg pembrolizumab;
(b) 100, 200, 300, 400, 800, or 1600 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² cisplatin; and
(d) 500 mg/m² pemetrexed;
wherein each of (a)-(d) is administered once every three weeks.

In certain embodiments, the human patient is administered:
(a) 200 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² cisplatin; and
(d) 500 mg/m² pemetrexed;
wherein each of (a)-(d) is administered once every three weeks.

In certain embodiments, the human patient is administered:
(a) 240 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² cisplatin; and
(d) 500 mg/m² pemetrexed;
wherein each of (a)-(d) is administered once every three weeks.

In certain embodiments, the human patient is administered:
(a) 2 mg/kg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² cisplatin; and
(d) 500 mg/m² pemetrexed;
wherein each of (a)-(d) is administered once every three weeks.

In certain embodiments, the human patient is administered:
(a) 400 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² cisplatin; and
(d) 500 mg/m² pemetrexed;
wherein (a) is administered once every six weeks, and each of (b)-(d) is administered once every three weeks.

In certain embodiments of various methods described herein, the human patient is administered paclitaxel at from about 50 mg/m² to about 200 mg/m² on Day 1, 8, and 15 of each three-week or four-week cycle.

In some embodiments of various methods described herein, the human patient is administered paclitaxel at 50 mg/m², 75 mg/m², 80 mg/m², 90 mg/m², 100 mg/m², 150 mg/m², or 200 mg/m² on Day 1, 8, and 15 of each three-week or four-week cycle.

In other embodiments of various methods described herein, the human patient is administered paclitaxel at 80 mg/m² on Day 1, 8, and 15 of each three-week cycle. In yet other embodiments, the human patient is administered paclitaxel at 90 mg/m² on Day 1, 8, and 15 of each four-week cycle.

Thus, in some embodiments, the human patient is administered:
(a) 200 mg, 240 mg, or 2 mg/kg pembrolizumab;
(b) 100, 200, 300, 400, 800, or 1600 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 80 mg/m² paclitaxel;
wherein each of (a)-(b) is administered once every three weeks, and wherein (c) is administered on Day 1, 8, and 15 of each three-week cycle.

In certain embodiments, the human patient is administered:
(a) 200 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 80 mg/m² paclitaxel;
wherein each of (a)-(b) is administered once every three weeks, and wherein (c) is administered on Day 1, 8, and 15 of each three-week cycle.

In certain embodiments, the human patient is administered:
(a) 240 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 80 mg/m² paclitaxel;
wherein each of (a)-(b) is administered once every three weeks, and wherein (c) is administered on Day 1, 8, and 15 of each three-week cycle.

In certain embodiments, the human patient is administered:
(a) 2 mg/kg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 80 mg/m² paclitaxel;
wherein each of (a)-(b) is administered once every three weeks, and wherein (c) is administered on Day 1, 8, and 15 of each three-week cycle.

In certain embodiments, the human patient is administered:
(a) 400 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 80 mg/m² paclitaxel;
wherein (a) is administered once every six weeks, (b) is administered once every three weeks, and (c) is administered on Day 1, 8, and 15 of each three-week cycle.

In some embodiments, the human patient is administered:
(a) 200 mg, 240 mg, or 2 mg/kg pembrolizumab;
(b) 100, 200, 300, 400, 800, or 1600 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 90 mg/m² paclitaxel;
wherein each of (a)-(b) is administered once every three weeks, and wherein (c) is administered on Day 1, 8, and 15 of each four-week cycle.

In certain embodiments, the human patient is administered:
(a) 200 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 90 mg/m² paclitaxel;
wherein each of (a)-(b) is administered once every three weeks, and wherein (c) is administered on Day 1, 8, and 15 of each four-week cycle.

In certain embodiments, the human patient is administered:
(a) 240 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 90 mg/m² paclitaxel;
wherein each of (a)-(b) is administered once every three weeks, and wherein (c) is administered on Day 1, 8, and 15 of each four-week cycle.

In certain embodiments, the human patient is administered:
(a) 2 mg/kg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 90 mg/m² paclitaxel;
wherein each of (a)-(b) is administered once every three weeks, and wherein (c) is administered on Day 1, 8, and 15 of each four-week cycle.

In certain embodiments, the human patient is administered:
(a) 400 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 90 mg/m² paclitaxel;
wherein (a) is administered once every six weeks, (b) is administered once every three weeks, and (c) is administered on Day 1, 8, and 15 of each four-week cycle.

In certain embodiments of various methods described herein, the human patient is administered docetaxel at from about 20 mg/m² to about 100 mg/m². In other embodiments, docetaxel is administered every week, every two weeks, or every three weeks.

In certain embodiments of various methods described herein, the human patient is administered docetaxel at 75 mg/m² every three weeks.

In some embodiments of various methods described herein, the human patient is administered docetaxel at 25 mg/m² every week.

Thus, in some embodiments, the human patient is administered:
(a) 200 mg, 240 mg, or 2 mg/kg pembrolizumab;
(b) 100, 200, 300, 400, 800, or 1600 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 25 or 75 mg/m² docetaxel;
wherein each of (a)-(b) is administered once every three weeks, and wherein (c) is administered at 25 mg/m² every week or 75 mg/m² every three weeks.

In certain embodiments, the human patient is administered:
(a) 200 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 25 or 75 mg/m² docetaxel;
wherein each of (a)-(b) is administered once every three weeks, and wherein (c) is administered at 25 mg/m² every week or 75 mg/m² every three weeks.

In certain embodiments, the human patient is administered:
(a) 240 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 25 or 75 mg/m² docetaxel;
wherein each of (a)-(b) is administered once every three weeks, and wherein (c) is administered at 25 mg/m² every week or 75 mg/m² every three weeks.

In certain embodiments, the human patient is administered:
(a) 2 mg/kg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 25 or 75 mg/m² docetaxel;
wherein each of (a)-(b) is administered once every three weeks, and wherein (c) is administered at 25 mg/m² every week or 75 mg/m² every three weeks.

In certain embodiments, the human patient is administered:
(a) 400 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 25 or 75 mg/m² docetaxel;
wherein each of (a)-(b) is administered once every three weeks, and wherein (c) is administered at 25 mg/m² every week or 75 mg/m² every three weeks.

In a specific embodiment, provided herein is a method of treating mesothelioma, comprising administering to a human patient in need thereof:
(a) 200 mg pembrolizumab;
(b) 800 mg of an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² cisplatin; and
(d) 500 mg/m² pemetrexed.

In certain embodiments of such a method, each of (a)-(d) is administered once every three weeks.

In some embodiments of such a method, (a)-(d) are administered on the same day; and each of (a)-(d) is administered sequentially, or two, three, or all of (a)-(d) are administered concurrently.

In a specific embodiment, provided herein is a method of treating TNBC, comprising administering to a human patient in need thereof:
(a) 200 mg pembrolizumab;
(b) 800 mg of an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 90 mg/m² paclitaxel.

In various embodiments of such a method, each of (a)-(b) is administered once every three weeks, and (c) is administered on Day 1, 8, and 15 of each four-week cycle.

In a specific embodiment, provided herein is a method of treating ovarian cancer, comprising administering to a human patient in need thereof:
(a) 200 mg pembrolizumab;
(b) 800 mg of an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 80 mg/m² paclitaxel.

In various embodiments of such a method, each of (a)-(b) is administered once every three weeks, and (c) is administered on Day 1, 8, and 15 of each three-week cycle.

In a specific embodiment, provided herein is a method of treating ovarian cancer, comprising administering to a human patient in need thereof:
(a) 200 mg pembrolizumab;
(b) 800 mg of an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 75 mg/m² docetaxel.

In various embodiments of such a method, each of (a)-(c) is administered once every three weeks.

In a specific embodiment, provided herein is a method of treating ovarian cancer, comprising administering to a human patient in need thereof:
(a) 200 mg pembrolizumab;
(b) 800 mg of an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) 25 mg/m² docetaxel.

In various embodiments of such a method, (a) and (b) are administered once every three weeks, and (c) is administered once every week.

### IV. BRIEF DESCRIPTION OF THE DRAWINGS

FIG. 1 illustrates a schema of a phase I, open label, multi-arm, multi-center study of MK-4830, in combination with pembrolizumab, cisplatin, and pemetrexed in patients with mesothelioma, in combination with pembrolizumab and paclitaxel in patients with TNBC or ovarian cancer.

### V. DETAILED DESCRIPTION OF THE INVENTION

### 1. Definitions

Certain technical and scientific terms are specifically defined below. Unless specifically defined elsewhere in this document, all other technical and scientific terms used herein have the meaning commonly understood by one of ordinary skill in the art to which this disclosure relates.

“About” when used to modify a numerically defined parameter (e.g., the dose of an anti-PD-1 antibody or antigen binding fragment thereof, an anti-ILT4 antibody or antigen binding fragment thereof, carboplatin, or pemetrexed, or the length of treatment time with a combination therapy described herein) means that the parameter is within 20%, within 15%, within 10%, within 9%, within 8%, within 7%, within 6%, within 5%, within 4%, within 3%, within 2%, within 1%, or less of the stated numerical value or range for that parameter; where appropriate, the stated parameter may be rounded to the nearest whole number. For example, a dose of about 5 mg/kg may vary between 4.5 mg/kg and 5.5 mg/kg.

As used herein, including the appended claims, the singular forms of words such as “a,” “an,” and “the,” include their corresponding plural references unless the context clearly dictates otherwise.

The terms “administration” or “administer” refers to the act of injecting or otherwise physically delivering a substance as it exists outside the body (e.g., an anti-PD-1 antibody, an anti-ILT4 antibody, carboplatin, and pemetrexed as described herein) into a patient, such as by mucosal, intradermal, intravenous, intramuscular delivery, and/or any other methods of physical delivery described herein or known in the art.

“PD-1 antagonist” means any chemical compound or biological molecule that blocks binding of PD-L1 to PD-1 and preferably also blocks binding of PD-L2 to PD-1. Alternative names or synonyms for PD-1 and its ligands include: PDCD1, PD1, CD279 and SLEB2 for PD-1; PDCDIL1, PDL1, B7H1, B7-4, CD274 and B7-H for PD-L1; and PDCDIL2, PDL2, B7-DC, Btdc and CD273 for PD-L2. In any of the treatment methods, medicaments and disclosed uses in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP 005009. Human PD-L1 and PD-L2 amino acid sequences can be found in NCBI Locus No.: NP 054862 and NP_079515, respectively.

“ILT4 antagonist” means any chemical compound or biological molecule that blocks binding of ILT4 to HLA-G, HLA-A, HLA-B, HLA-F, or angiopoietin-like protein (ANGPTL, such as ANGPTL1, ANGPTL4, or ANGPTL7). Alternative names or synonyms for ILT4 and its ligands include but are not limited to: ILT-4, leukocyte immunoglobulin-like receptor subfamily B member 2 (LILRB2), MIR10, MIR-10, LIR2, LIR-2, CD85D for ILT4; MHC-G or major histocompatibility complex, class I, G for HLA-G; major histocompatibility complex, class I, A for HLA-A; AS, B-4901, major histocompatibility complex, class I, B for HLA-B; CDA12, HLA-CDA12, or major histocompatibility complex, class I, F for HLA-F; angiopoietin-3, ANG3, ANGPT3, ARP1, UNQ162, angiopoietin like 1 for ANGPTL1; ARP4, HFARP, PGAR, UNQ171, angiopoietin like 4 for ANGPTL4; and CDT6, angiopoietin like 7 for ANGPTL7. In any of the treatment methods, medicaments and disclosed uses in which a human individual is being treated, the ILT4 antagonist blocks binding of human ILT4 to human HLA-G, HLA-A, HLA-B, HLA-F, ANGPTL1, ANGPTL4, or ANGPTL7. Human ILT4 precursor amino acid sequence can be found in NCBI Locus No.: AAB88119.1. Human HLA-G, HLA-A, HLA-B, and HLA-F precursor amino acid sequences can be found in NCBI Locus No.: P17693.1, P04439.2, P01889.3, P30511.3, respectively. Human ANGPTL1, ANGPTL4, and ANGPTL7 precursor amino acid sequences can be found in NCBI Locus No.: NP_001363692, Q9BY76.2, and O43827.1, respectively.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} B-sheet framework, or one of three hypervariable regions (L1, L2, or L3) within the non-framework region of the antibody V_{L} B-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

As used herein, unless otherwise indicated, “antibody fragment” or “antigen binding fragment” refers to a fragment of an antibody that retains the ability to bind specifically to the antigen, e.g., fragments that retain one or more CDR regions. An antibody that “specifically binds to” PD-1 or ILT4 is an antibody that exhibits preferential binding to PD-1 or ILT4 (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., PD-1 or ILT4). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, the terms “at least one” item or “one or more” item each include a single item selected from the list as well as mixtures of two or more items selected from the list.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

The term “subject” (alternatively “patient”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorphs (e.g., rabbits), rodents (e.g., rats or mice), Procyon lotor (e.g., raccoons). In particular embodiments, the subject is human. The term “subject in need thereof” as used herein refers to a subject diagnosed with or suspected of having cancer or an infectious disease as defined herein.

“Biotherapeutic agent” means a cell (such as a CAR-T cell), a vaccine (such as an anti-tumor vaccine), a biological molecule (such as an antibody, antibody-drug conjugate, fusion protein, peptide, nucleic acid, etc.), that enhances anti-tumor immune response and/or suppresses tumor growth.

“Chemotherapeutic agent” refers to a chemical substance that can cause death of cancer cells, or interfere with growth, division, repair, and/or function of cancer cells. Classes of chemotherapeutic agents include but are not limited to: alkylating agents, antimetabolites, plant alkaloids, antitumor antibiotics, topisomerase inhibitors, etc.

“Alkylating agent” refers to a compound that inhibits proliferation of cancer cells by adding an alkyl group to the guanine base of the DNA molecule of the cancer cells. Commonly used alkylating agents include, but are not limited to, 5-azacytidine, decitabine, temozolomide, dactinomycin (also known as actinomycin-D), melphalan, altretamine, carmustine, bendamustine, busulfan, carboplatin, lomustine, cisplatin, chlorambucil, cyclophosphamide, dacarbazine, altretamine, ifosfamide, procarbazine, mechlorethamine, streptozocin, thiotepa, or pharmaceutically acceptable salts thereof.

“Antimetabolite” refers to a compound that inhibits the use of a metabolite by cancer cells. In certain embodiments, the antimetabolite interferes with one or more enzymes or their reactions that are necessary for DNA synthesis. In some embodiments, the antimetabolite acts as a substitute to an actual metabolite that would be used in the normal metabolism. Commonly used antimetabolites include, but are not limited to, claribine, 5-fluorouracil, 6-thioguanine, cytarabine (also known as arabinosylcytosine (Ara-C)), cytarabine liposomal (also known as Liposomal Ara-C, sold under the tradename DEPOCYT™), decitabine (sold under the tradename DACOGEN®), hydroxyurea and fludarabine, floxuridine, cladribine (also known as 2-chlorodeoxyadenosine (2-CdA), methotrexate (also known as amethopterin, methotrexate sodium (MTX)), pemetrexed, and pentostatin.

“Mitosis inhibitor” refers to a compound that inhibits mitosis or division of cells. In certain embodiments, the mitosis inhibitors target tubulin. Some mitosis inhibitors bind to tubulin and inhibit microtubule assembly, whereas other mitosis inhibitors stabilize the microtubule polymer and protect it from disassembly. Non-limiting examples of mitosis inhibitors include eribulin, docetaxel, paclitaxel, vincristine, teniposide, etoposide, vinblastine, vinorelbine, cabazitaxel, ixabepilone, and estramustine.

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient, and can be readily selected by a person skilled in the art.

The term “variant” when used in relation to an antibody (e.g., an anti-PD-1 antibody or an anti-ILT4 antibody) or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified anti-PD-1 antibody. Variants may be naturally occurring or may be artificially constructed. Polypeptide variants may be prepared from the corresponding nucleic acid molecules encoding the variants. In specific embodiments, an antibody variant (e.g., an anti-PD-1 antibody variant or an anti-ILT4 antibody variant) at least retains the antibody functional activity. In specific embodiments, an anti-PD-1 antibody variant binds to PD-1 and/or is antagonistic to PD-1 activity. In some embodiments, an anti-ILT4 antibody variant binds to ILT4 and/or is antagonistic to ILT4 activity.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“RECIST 1.1 Response Criteria” as used herein means the definitions set forth in Eisenhauer, E. A. et al., Eur. J. Cancer 45:228-247 (2009) for target lesions or nontarget lesions, as appropriate based on the context in which response is being measured.

“Sustained response” means a sustained therapeutic effect after cessation of treatment as described herein. In some embodiments, the sustained response has a duration that is at least the same as the treatment duration, or at least 1.5, 2.0, 2.5 or 3 times longer than the treatment duration.

“Treat” or “treating” cancer as used herein means to administer a therapeutic combination of a PD-1 antagonist, an ILT4 antagonist, and one or more chemotherapeutic agents, to a subject having cancer or diagnosed with cancer to achieve at least one positive therapeutic effect, such as, for example, reduced number of cancer cells, reduced tumor size, reduced rate of cancer cell infiltration into peripheral organs, or reduced rate of tumor metastasis or tumor growth. Such “treatment” may result in a slowing, interrupting, arresting, controlling, or stopping of the progression of cancer as described herein but does not necessarily indicate a total elimination of the cancer or the symptoms of the cancer. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination therapy of the disclosure is any of PR, CR, OR, PFS, DFS, and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination therapy of the disclosure is any of PR, CR, PFS, DFS, or OR that is assessed using RECIST 1.1 response criteria. The treatment regimen for a combination therapy of the disclosure that is effective to treat a cancer patient may vary according to factors such as the disease state, age, and weight of the patient, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

As used herein, the terms “combination therapy” and “therapeutic combination” refer to treatments in which an anti-human PD-1 monoclonal antibody or antigen-binding fragment thereof, an anti-human ILT4 monoclonal antibody or antigen-binding fragment thereof, one or more chemotherapeutic agents, and optionally additional therapeutic agents, each are administered to a patient in a coordinated manner, over an overlapping period of time. The period of treatment with the anti-human PD-1 monoclonal antibody (or antigen-binding fragment thereof) (the “anti-PD-1 treatment”) is the period of time that a patient undergoes treatment with the anti-human PD-1 monoclonal antibody (or antigen-binding fragment thereof); that is, the period of time from the initial dosing with the anti-human PD-1 monoclonal antibody (or antigen-binding fragment thereof) through the final day of a treatment cycle. Similarly, the period of treatment with the anti-human ILT4 monoclonal antibody (or antigen-binding fragment thereof) (the “anti-ILT4 treatment”) is the period of time that a patient undergoes treatment with the anti-human ILT4 monoclonal antibody (or antigen-binding fragment thereof); that is, the period of time from the initial dosing with the anti-human ILT4 monoclonal antibody (or antigen-binding fragment thereof) through the final day of a treatment cycle. The period of treatment with chemotherapeutic agent (the “chemotherapy treatment”) is the period of time that a patient undergoes treatment with the chemotherapeutic agent; that is, the period of time from the initial dosing with the chemotherapeutic agent through the final day of a treatment cycle. In the methods and therapeutic combinations described herein, the anti-PD-1 treatment overlaps by at least one day with the anti-ILT4 treatment and overlaps by at least one day with the chemotherapy treatment. In certain embodiments, the anti-PD-1 treatment, the anti-ILT4 treatment, and the chemotherapy treatment are the same period of time. In some embodiments, the anti-PD-1 treatment begins prior to the anti-ILT4 and/or the chemotherapy treatment. In other embodiments, the anti-PD-1 treatment begins after the anti-ILT4 and/or the chemotherapy treatment. In yet other embodiments, the anti-ILT4 treatment begins prior to the anti-PD-1 and/or the chemotherapy treatment. In still other embodiments, the anti-ILT4 treatment begins after the anti-PD-1 and/or the chemotherapy treatment. In some embodiments, the chemotherapy treatment begins prior to the anti-ILT4 and/or the anti-PD-1 treatment. In other embodiments, the chemotherapy treatment begins after the anti-ILT4 and/or the anti-PD-1 treatment. In certain embodiments, the anti-PD-1 treatment is terminated prior to termination of the anti-ILT4 and/or the chemotherapy treatment. In other embodiments, the anti-PD-1 treatment is terminated after termination of the anti-ILT4 and/or the chemotherapy treatment. In yet other embodiments, the anti-ILT4 treatment is terminated prior to termination of the anti-PD-1 and/or the chemotherapy treatment. In still other embodiments, the anti-ILT4 treatment is terminated after termination of the anti-PD-1 and/or the chemotherapy treatment. In certain embodiments, the chemotherapy treatment is terminated prior to termination of the anti-ILT4 and/or the anti-PD-1 treatment. In other embodiments, the chemotherapy treatment is terminated after termination of the anti-ILT4 and/or the anti-PD-1 treatment.

The terms “treatment regimen,” “dosing protocol,” and “dosing regimen” are used interchangeably to refer to the dose and timing of administration of each therapeutic agent in a combination therapy of the disclosure.

“Tumor” as it applies to a subject diagnosed with, or suspected of having, a cancer refers to a malignant or potentially malignant neoplasm or tissue mass of any size, and includes primary tumors and secondary neoplasms. Non-limiting examples of tumors include solid tumor (e.g., sarcoma (such as chondrosarcoma), carcinoma (such as colon carcinoma), blastoma (such as hepatoblastoma), etc.) and blood tumor (e.g., leukemia (such as acute myeloid leukemia (AML)), lymphoma (such as DLBCL), multiple myeloma (MM), etc.).

The term “tumor volume” or “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of #1%, +2%, +3%, +4%, +5%, +10%, +15%, and +20% and their numerical equivalents. All ranges also are intended to include all included sub-ranges, although not necessarily explicitly set forth. For example, a range of 3 to 7 days is intended to include 3, 4, 5, 6, and 7 days. In addition, the term “or,” as used herein, denotes alternatives that may, where appropriate, be combined; that is, the term “or” includes each listed alternative separately as well as their combination.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

### 2. PD-1 Antagonist

Provided herein are PD-1 antagonists that can be used in the various methods, kits, and uses disclosed herein, including any chemical compound or biological molecule that blocks binding of PD-L1 to PD-1 and preferably also blocks binding of PD-L2 to PD-1.

Any monoclonal antibodies that bind to a PD-1 polypeptide, a PD-1 polypeptide fragment, a PD-1 peptide, or a PD-1 epitope and block the interaction between PD-1 and its ligand PD-L1 or PD-L2 can be used. In some embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1. In other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L2. In yet other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1 and the interaction between PD-1 and PD-L2.

Any monoclonal antibodies that bind to a PD-L1 polypeptide, a PD-L1 polypeptide fragment, a PD-L1 peptide, or a PD-L1 epitope and block the interaction between PD-L1 and PD-1 can also be used.

In certain embodiments, the anti-human PD-1 monoclonal antibody is selected from the group consisting of pembrolizumab, nivolumab, cemiplimab, pidilizumab (U.S. Pat. No. 7,332,582), AMP-514 (MedImmune LLC, Gaithersburg, MD), PDR001 (U.S. Pat. No. 9,683,048), BGB-A317 (U.S. Pat. No. 8,735,553), and MGA012 (MacroGenics, Rockville, MD). In one embodiment, the anti-human PD-1 monoclonal antibody is pembrolizumab. In another embodiment, the anti-human PD-1 monoclonal antibody is nivolumab. In another embodiment, the anti-human PD-1 monoclonal antibody is cemiplimab. In yet another embodiment, the anti-human PD-1 monoclonal antibody is pidilizumab. In one embodiment, the anti-human PD-1 monoclonal antibody is AMP-514. In another embodiment, the anti-human PD-1 monoclonal antibody is PDR001. In yet another embodiment, the anti-human PD-1 monoclonal antibody is BGB-A317. In still another embodiment, the anti-human PD-1 monoclonal antibody is MGA012.

In some embodiments, the anti-human PD-1 monoclonal antibody can be any antibody, antigen binding fragment thereof, or variant thereof disclosed in U.S. Pat. Nos. 7,488,802, 7,521,051, 8,008,449, 8,354,509, 8,168,757, WO2004/004771, WO2004/072286, WO2004/056875, US2011/0271358, and WO 2008/156712, the disclosures of which are incorporated by reference herein in their entireties.

Examples of monoclonal antibodies that bind to human PD-L1 that can be used in various methods, kits, and uses described herein are disclosed in WO2013/019906, WO2010/077634, and U.S. Pat. No. 8,383,796, the disclosures of which are incorporated by reference herein in their entireties. Specific anti-human PD-L1 monoclonal antibodies useful as the PD-1 antagonist in the various methods, kits, and uses described include atezolizumab, durvalumab, avelumab, BMS-936559, and an antibody comprising the heavy chain and light chain variable regions of SEQ ID NO:20 and SEQ ID NO:21, respectively, of WO2013/019906.

Other PD-1 antagonists useful in various methods, kits, and uses described herein include an immunoadhesin molecule that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342, the disclosures of which are incorporated by reference herein in their entireties. Specific fusion proteins useful as the PD-1 antagonist in various methods, kits, and uses described herein include AMP-224 (also known as B7-DCIg), which is a PD-L2-Fc fusion protein and binds to human PD-1.

In various embodiments, the anti-human PD-1 or anti-human PD-L1 monoclonal antibody or antigen binding fragment thereof comprises a variant of the amino acid sequences of the anti-human PD-1 or anti-human PD-L1 antibodies described herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human PD-1 or anti-human PD-L1 monoclonal antibody or antigen binding fragment thereof has a V_{L} domain with at least 95%, 90%, 85%, 80%, 75% or 50% sequence homology to one of the V_{L} domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a V_{H} domain with at least 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has a V_{L} domain with at least 95%, 90%, 85%, 80%, 75% or 50% sequence homology to one of the V_{L} domains of the anti-human PD-1 or anti-human PD-L1 antibodies described herein and a V_{H} domain with at least 95%, 90%, 85%, 80%, 75% or 50% sequence homology to one of the V_{H} domains of the anti-human PD-1 or anti-human PD-L1 antibodies described herein, and exhibits specific binding to PD-1 or PD-L1.

In one embodiment, the anti-human PD-1 or anti-human PD-L1 monoclonal antibody or antigen binding fragment thereof has a V_{L} domain having up to 1, 2, 3, 4, 5 or more amino acid substitutions, deletions and/or additions in one of the V_{L} domains of the anti-human PD-1 or anti-human PD-L1 antibodies described herein, and exhibits specific binding to PD-1 or PD-L1. In another embodiment, the anti-human PD-1 or anti-human PD-L1 monoclonal antibody or antigen binding fragment thereof has a V_{H} domain having up to 1, 2, 3, 4, 5 or more amino acid substitutions, deletions, and/or additions in one of the V_{H} domains of the anti-human PD-1 or anti-human PD-L1 antibodies described herein, and exhibits specific binding to PD-1 or PD-L1. In yet another embodiment, the anti-human PD-1 or anti-human PD-L1 monoclonal antibody or antigen binding fragment thereof has a V_{L} domain having up to 1, 2, 3, 4, 5 or more amino acid substitutions, deletions, and/or additions in one of the V_{L} domains of the anti-human PD-1 or anti-human PD-L1 antibodies described herein and a V_{H} domain having up to 1, 2, 3, 4, 5 or more amino acid substitutions, deletions, and/or additions in one of the V_{H} domains of the anti-human PD-1 or anti-human PD-L1 antibodies described herein, and exhibits specific binding to PD-1 or PD-L1.

In various embodiments, the anti-human PD-1 or anti-human PD-L1 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-human PD-1 or anti-human PD-L1 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-human PD-1 or anti-human PD-L1 monoclonal antibody is a human antibody. In other embodiments, the anti-human PD-1 or anti-human PD-L1 monoclonal antibody is a humanized antibody.

In some embodiments, the light chain of the anti-human PD-1 or anti-human PD-L1 monoclonal antibody has a human kappa backbone. In other embodiments, the light chain of the anti-human PD-1 or anti-human PD-L1 monoclonal antibody has a human lambda backbone.

In some embodiments, the heavy chain of the anti-human PD-1 or anti-human PD-L1 monoclonal antibody has a human IgG1 backbone. In other embodiments, the heavy chain of the anti-human PD-1 or anti-human PD-L1 monoclonal antibody has a human IgG2 backbone. In yet other embodiments, the heavy chain of the anti-human PD-1 or anti-human PD-L1 monoclonal antibody has a human IgG3 backbone. In still other embodiments, the heavy chain of the anti-human PD-1 or anti-human PD-L1 monoclonal antibody has a human IgG4 backbone.

In some embodiments, the heavy chain of the anti-human PD-1 or anti-human PD-L1 monoclonal antibody has a human IgG1 variant backbone. In other embodiments, the heavy chain of the anti-human PD-1 or anti-human PD-L1 monoclonal antibody has a human IgG2 variant backbone. In yet other embodiments, the heavy chain of the anti-human PD-1 or anti-human PD-L1 monoclonal antibody has a human IgG3 variant backbone. In still other embodiments, the heavy chain of the anti-human PD-1 or anti-human PD-L1 monoclonal antibody has a human IgG4 variant (e.g., IgG4 with S228P mutation) backbone.

### 3. ILT4 Antagonist

Also provided herein are ILT4 antagonists that can be used in the various methods, kits, and uses disclosed herein, including any chemical compound or biological molecule that blocks binding of ILT4 to HLA-G, HLA-A, HLA-B, HLA-F, and/or ANGPTL (such as ANGPTL1, ANGPTL4, or ANGPTL7).

Any monoclonal antibodies that bind to an ILT4 polypeptide, an ILT4 polypeptide fragment, an ILT4 peptide, or an ILT4 epitope and block the interaction between ILT4 and HLA-G, HLA-A, HLA-B, HLA-F, and/or ANGPTL (such as ANGPTL1, ANGPTL4, or ANGPTL7) can be used.

In certain embodiments of various methods, kits, or uses provided herein, the anti-human ILT4 monoclonal antibody or antigen binding fragment thereof comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively.

In some embodiments of various methods, kits, or uses provided herein, the anti-human ILT4 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9.

In other embodiments of various methods, kits, or uses provided herein, the anti-human ILT4 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

In some embodiments, the anti-human ILT4 monoclonal antibody can be any antibody, antigen binding fragment thereof, or variant thereof disclosed in WO 2018/187518 and WO 2019/126514, the disclosures of which are incorporated by reference herein in their entireties.

In various embodiments, the anti-human ILT4 monoclonal antibody or antigen binding fragment thereof comprises a variant of the amino acid sequences of the anti-ILT4 antibodies disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human ILT4 monoclonal antibody or antigen binding fragment thereof has a V_{L} domain with at least 95%, 90%, 85%, 80%, 75% or 50% sequence homology to one of the V_{L} domains of the anti-ILT4 antibodies described herein, and exhibits specific binding to ILT4. In another embodiment, the anti-human ILT4 monoclonal antibody or antigen binding fragment thereof has a V_{H} domain with at least 95%, 90%, 85%, 80%, 75% or 50% sequence homology to one of the V_{H} domains of the anti-ILT4 antibodies described herein, and exhibits specific binding to ILT4. In yet another embodiment, the anti-human ILT4 monoclonal antibody or antigen binding fragment thereof has a V_{L} domain with at least 95%, 90%, 85%, 80%, 75% or 50% sequence homology to one of the V_{L} domains of the anti-ILT4 antibodies described herein and a V_{H} domain with at least 95%, 90%, 85%, 80%, 75% or 50% sequence homology to one of the V_{H} domains of the anti-ILT4 antibodies described herein, and exhibits specific binding to ILT4.

In one embodiment, the anti-human ILT4 monoclonal antibody or antigen binding fragment thereof has a V_{L} domain having up to 1, 2, 3, 4, 5 or more amino acid substitutions, deletions and/or additions in one of the V_{L} domains of the anti-ILT4 antibodies described herein, and exhibits specific binding to ILT4. In another embodiment, the anti-human ILT4 monoclonal antibody or antigen binding fragment thereof has a V_{H} domain having up to 1, 2, 3, 4, 5 or more amino acid substitutions, deletions, and/or additions in one of the V_{H} domains of the anti-ILT4 antibodies described herein, and exhibits specific binding to ILT4. In yet another embodiment, the anti-human ILT4 monoclonal antibody or antigen binding fragment thereof has a V_{L} domain having up to 1, 2, 3, 4, 5 or more amino acid substitutions, deletions, and/or additions in one of the V_{L} domains of the anti-ILT4 antibodies described herein and a V_{H} domain having up to 1, 2, 3, 4, 5 or more amino acid substitutions, deletions, and/or additions in one of the V_{H} domains of the anti-ILT4 antibodies described herein, and exhibits specific binding to ILT4.

In various embodiments, the anti-human ILT4 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-ILT4 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-human ILT4 monoclonal antibody is a human antibody. In other embodiments, the anti-human ILT4 monoclonal antibody is a humanized antibody.

In some embodiments, the light chain of the anti-human ILT4 monoclonal antibody has a human kappa backbone. In other embodiments, the light chain of the anti-human ILT4 monoclonal antibody has a human lambda backbone.

In some embodiments, the heavy chain of the anti-human ILT4 monoclonal antibody has a human IgG1 backbone. In other embodiments, the heavy chain of the anti-human ILT4 monoclonal antibody has a human IgG2 backbone. In yet other embodiments, the heavy chain of the anti-human ILT4 monoclonal antibody has a human IgG3 backbone. In still other embodiments, the heavy chain of the anti-human ILT4 monoclonal antibody has a human IgG4 backbone.

In some embodiments, the heavy chain of the anti-human ILT4 monoclonal antibody has a human IgG1 variant backbone. In other embodiments, the heavy chain of the anti-human ILT4 monoclonal antibody has a human IgG2 variant backbone. In yet other embodiments, the heavy chain of the anti-human ILT4 monoclonal antibody has a human IgG3 variant backbone. In still other embodiments, the heavy chain of the anti-human ILT4 monoclonal antibody has a human IgG4 variant (e.g., IgG4 with S228P mutation) backbone.

In certain embodiments, the ILT4 antagonist is a molecule that binds to HLA-G, HLA-A, HLA-B, HLA-F, ANGPTL1, ANGPTL4, or ANGPTL7 and blocks the binding of ILT4 to HLA-G, HLA-A, HLA-B, HLA-F, ANGPTL1, ANGPTL4, or ANGPTL7. In one embodiment, the ILT4 antagonist is a molecule that binds to HLA-G and blocks the binding of ILT4 to HLA-G. In one embodiment, the ILT4 antagonist is a molecule that binds to HLA-A and blocks the binding of ILT4 to HLA-A. In one embodiment, the ILT4 antagonist is a molecule that binds to HLA-B and blocks the binding of ILT4 to HLA-B. In one embodiment, the ILT4 antagonist is a molecule that binds to HLA-F and blocks the binding of ILT4 to HLA-F. In one embodiment, the ILT4 antagonist is a molecule that binds to ANGPTL1 and blocks the binding of ILT4 to ANGPTL1. In one embodiment, the ILT4 antagonist is a molecule that binds to ANGPTL4 and blocks the binding of ILT4 to ANGPTL4. In one embodiment, the ILT4 antagonist is a molecule that binds to ANGPTL7 and blocks the binding of ILT4 to ANGPTL7. In some embodiments, the molecule that binds to HLA-G, HLA-A, HLA-B, HLA-F, ANGPTL1, ANGPTL4, or ANGPTL7 is a monoclonal antibody specifically binding to HLA-G, HLA-A, HLA-B, HLA-F, ANGPTL1, ANGPTL4, or ANGPTL7.

### 4. Additional Therapeutic Agents

The PD-1 antagonists and the ILT4 antagonists can be used with additional therapeutic agents in the various methods, kits, and uses disclosed herein.

The additional therapeutic agent can be, e.g., a chemotherapeutic or a biotherapeutic agent (including but not limited to antibodies or antigen binding fragments thereof that specifically bind to an antigen selected from the group consisting of: PD-L1, PD-L2, CTLA4, BTLA, TIM3, HVEM, GITR, CD27, ILT2, ILT3, ILT5, SIRPα, NKG2A, NKG2C, NKG2E, TSLP, IL10, VISTA, VEGF, EGFR, Her2/neu, VEGF receptors, other growth factor receptors, CD20, CD28, CD40, CD-40L, CD70, OX-40, 4-1BB, and ICOS).

The additional therapeutic agent can be selected from the group consisting of STING agonists, poly ADP ribose polymerase (PARP) inhibitors, mitogen-activated protein kinase (MEK) inhibitors, cyclin-dependent kinase (CDK) inhibitors, indoleamine 2,3-dioxygenase (IDO) inhibitors, tryptophan 2,3-dioxygenase (TDO) selective inhibitors, anti-viral compounds, antigens, adjuvants, anti-cancer agents, CTLA-4 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anti-cancer vaccines.

The additional therapeutic agent can be an anti-viral compound, including but not limited to, hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors.

The additional therapeutic agent can be a cytotoxic agent, including but not limited to, arsenic trioxide (sold under the tradename TRISENOXR) and asparaginase (also known as L-asparaginase and Erwinia L-asparaginase, sold under the tradenames ELSPARR and KIDROLASER).

The additional therapeutic agent can be an chemotherapeutic agent, including but not limited to, abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-prolyl-1-Lproline-t-butylamide (SEQ ID NO: 11), cachectin, cemadotin, chlorambucil, cyclophosphamide, 3′,4′-didehydro-4′deoxy-8′-norvin-caleukoblastine, dinaciclib, docetaxol, doxetaxel, cyclophosphamide, carmustine, carboplatin, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyurea andtaxanes, ifosfamide, liarozole, lonidamine, lomustine, MDV3100, mechlorethamine (nitrogen mustard), melphalan, mivobulin isethionate, rhizoxin, sertenef, streptozocin, mitomycin, methotrexate, taxanes, nilutamide, olaparib, onapristone, paclitaxel, pemetrexed, prednimustine, procarbazine, RPR109881, selumetinib, stramustine phosphate, tamoxifen, tasonermin, taxol, tretinoin, vinblastine, vincristine, vindesine sulfate, and vinflunine, and pharmaceutically acceptable salts thereof.

The additional therapeutic agent can be a vascular endothelial growth factor (VEGF) receptor inhibitors, including but not limited to, bevacizumab (sold under the trademark AVASTIN by Genentech/Roche), axitinib (described in PCT International Patent Publication No. WO01/002369), Brivanib Alaninate ((S)-((R)-1-(4-(4-Fluoro-2-methyl-1H-indol-5-yloxy)-5-methylpyrrolo[2,1-f][1,2,4]triazin-6-yloxy) propan-2-yl) 2-aminopropanoate, also known as BMS-582664), motesanib (N-(2,3-dihydro-3,3-dimethyl-1H-indol-6-yl)-2-[(4-pyridinylmethyl)amino]-3-pyridinecarboxamide. and described in PCT International Patent Application Publication No. WO02/068470), pasireotide (also known as SO 230, and described in PCT International Patent Publication No. WO02/010192), and sorafenib.

The additional therapeutic agent can be a topoisomerase II inhibitor, including but not limited to, etoposide and teniposide.

The additional therapeutic agent can be an alkylating agent, including but not limited to, 5-azacytidine, decitabine, temozolomide, dactinomycin (also known as actinomycin-D, melphalan, altretamine, carmustine, bendamustine, busulfan, carboplatin, lomustine, cisplatin, chlorambucil, cyclophosphamide, dacarbazine, altretamine, ifosfamide, procarbazine, mechlorethamine, streptozocin, thiotepa, and pharmaceutically acceptable salts thereof.

The additional therapeutic agent can be an anti-tumor antibiotic, including but not limited to, doxorubicin, bleomycin, daunorubicin liposomal (daunorubicin citrate liposome), mitoxantrone, epirubicin, idarubicin, and mitomycin C.

The additional therapeutic agent can be an antimetabolite, including but not limited to, claribine, 5-fluorouracil, 6-thioguanine, cytarabine (also known as arabinosylcytosine (Ara-C)), cytarabine liposomal (also known as Liposomal Ara-C, sold under the tradename DEPOCYT™), decitabine (sold under the tradename DACOGEN®), hydroxyurea and fludarabine, floxuridine, cladribine (also known as 2-chlorodeoxyadenosine (2-CdA), methotrexate (also known as amethopterin, methotrexate sodium (MTX)), pemetrexed, and pentostatin.

The additional therapeutic agent can be an mitosis inhibitor, including but not limited to, eribulin, docetaxel, paclitaxel, vincristine, teniposide, etoposide, vinblastine, vinorelbine, cabazitaxel, ixabepilone, and estramustine.

The additional therapeutic agent can be a retinoid, including but not limited to, alitretinoin, tretinoin, isotretinoin, and bexarotene.

In a specific embodiment, the additional therapeutic agents used with a PD-1 antagonist and an ILT4 antagonist are an alkylating agent and an antimetabolite. In another specific embodiment, the additional therapeutic agents used with a PD-1 antagonist and an ILT4 antagonist are cisplatin and pemetrexed.

In another specific embodiment, the additional therapeutic agent used with a PD-1 antagonist and an ILT4 antagonist is a mitosis inhibitor. In yet another specific embodiment, the additional therapeutic agent used with a PD-1 antagonist and an ILT4 antagonist is paclitaxel. In yet another specific embodiment, the additional therapeutic agent used with a PD-1 antagonist and an ILT4 antagonist is docetaxel.

### 5. Methods of Treating Cancer Using a Combination of a PD-1 Antagonist, an ILT4 Antagonist, and One or More Chemotherapeutic Agents

In another aspect, provided herein are methods of treating cancer (e.g., mesothelioma, ovarian cancer, or TNBC) using a combination of a PD-1 antagonist, an ILT4 antagonist, and one or more additional chemotherapeutic agents (e.g., cisplatin, pemetrexed, paclitaxel, or docetaxel).

In certain embodiments, the method of treating cancer comprises administering to a human patient in need thereof:
(a) a PD-1 antagonist;
(b) an ILT4 antagonist;
(c) cisplatin; and
(d) pemetrexed.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., NSCLC), pleural mesothelioma, gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In some embodiments, the cancer is relapsed. In other embodiments, the cancer is refractory. In yet other embodiments, the cancer is relapsed and refractory.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is NSCLC. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelfibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer.

In one embodiment, the cancer is mesothelioma. In another embodiment, the mesothelioma is advanced mesothelioma. In yet another embodiment, the mesothelioma is recurrent mesothelioma.

In certain embodiments, provided herein is a method of treating mesothelioma, comprising administering to a human patient in need thereof:
(a) a PD-1 antagonist;
(b) an ILT4 antagonist;
(c) cisplatin; and
(d) pemetrexed.

In certain embodiments, provided herein is a method of treating advanced mesothelioma, comprising administering to a human patient in need thereof:
(a) a PD-1 antagonist;
(b) an ILT4 antagonist;
(c) cisplatin; and
(d) pemetrexed.

In certain embodiments, provided herein is a method of treating recurrent mesothelioma, comprising administering to a human patient in need thereof:
(a) a PD-1 antagonist;
(b) an ILT4 antagonist;
(c) cisplatin; and
(d) pemetrexed.

In another aspect, provided herein is a method of treating cancer, comprising administering to a human patient in need thereof:
(a) a PD-1 antagonist;
(b) an ILT4 antagonist; and
(c) a mitosis inhibitor.

In one embodiment, the mitosis inhibitor is paclitaxel. In another embodiment, the mitosis inhibitor is docetaxel.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., NSCLC), pleural mesothelioma, gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In some embodiments, the cancer is relapsed. In other embodiments, the cancer is refractory. In yet other embodiments, the cancer is relapsed and refractory.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In yet still another embodiment, the cancer is TNBC. In still another embodiment, the cancer is prostate cancer. In one embodiment, the cancer is bone cancer. In another embodiment, the cancer is lung cancer. In yet another embodiment, the cancer is NSCLC. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelfibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer.

In certain embodiments, provided herein is a method of treating breast cancer, comprising administering to a human patient in need thereof:
(a) a PD-1 antagonist;
(b) an ILT4 antagonist;
(c) paclitaxel.

In certain embodiments, provided herein is a method of treating TNBC, comprising administering to a human patient in need thereof:
(a) a PD-1 antagonist;
(b) an ILT4 antagonist;
(c) paclitaxel.

In certain embodiments, provided herein is a method of treating ovarian cancer, comprising administering to a human patient in need thereof:
(a) a PD-1 antagonist;
(b) an ILT4 antagonist;
(c) paclitaxel.

In certain embodiments, provided herein is a method of treating ovarian cancer, comprising administering to a human patient in need thereof:
(a) a PD-1 antagonist;
(b) an ILT4 antagonist;
(c) docetaxel.

In certain embodiments, the method of treating cancer comprises administering to a human patient in need thereof:
(a) a PD-1 antagonist as disclosed in Section V.2;
(b) an ILT4 antagonist as disclosed in Section V.3; and
(c) one or more therapeutic agents as disclosed in Section V.4.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

In certain embodiments, the PD-1 antagonist is an anti-human PD-L1 monoclonal antibody or antigen binding fragment thereof. In some embodiments, the anti-human PD-L1 monoclonal antibody is a human antibody. In other embodiments, the anti-human PD-L1 monoclonal antibody is a humanized antibody.

In certain embodiments, the ILT4 antagonist is an anti-human ILT4 monoclonal antibody or antigen binding fragment thereof. In some embodiments, the anti-human ILT4 monoclonal antibody is a human antibody. In other embodiments, the anti-human ILT4 monoclonal antibody is a humanized antibody.

Thus, in certain embodiments, provided herein is a method for treating cancer, comprising administering to a human patient in need thereof:
(a) a human or humanized anti-human PD-1 monoclonal antibody or antigen binding fragment thereof;
(b) a human or humanized anti-human ILT4 monoclonal antibody or antigen binding fragment thereof;
(c) cisplatin; and
(d) pemetrexed.

In some embodiments, provided herein is a method for treating cancer, comprising administering to a human patient in need thereof:
(a) a human anti-human PD-1 monoclonal antibody or antigen binding fragment thereof;
(b) a human anti-human ILT4 monoclonal antibody or antigen binding fragment thereof;
(c) cisplatin; and
(d) pemetrexed.

In other embodiments, provided herein is a method for treating cancer, comprising administering to a human patient in need thereof:
(a) a humanized anti-human PD-1 monoclonal antibody or antigen binding fragment thereof;
(b) a humanized anti-human ILT4 monoclonal antibody or antigen binding fragment thereof;
(c) cisplatin; and
(d) pemetrexed.

In certain embodiments, provided herein is a method for treating cancer, comprising administering to a human patient in need thereof:
(a) a human or humanized anti-human PD-1 monoclonal antibody or antigen binding fragment thereof;
(b) a human or humanized anti-human ILT4 monoclonal antibody or antigen binding fragment thereof; and
(c) paclitaxel.

In some embodiments, provided herein is a method for treating cancer, comprising administering to a human patient in need thereof:
(a) a human anti-human PD-1 monoclonal antibody or antigen binding fragment thereof;
(b) a human anti-human ILT4 monoclonal antibody or antigen binding fragment thereof; and
(c) paclitaxel.

In other embodiments, provided herein is a method for treating cancer, comprising administering to a human patient in need thereof:
(a) a humanized anti-human PD-1 monoclonal antibody or antigen binding fragment thereof;
(b) a humanized anti-human ILT4 monoclonal antibody or antigen binding fragment thereof; and
(c) paclitaxel.

In certain embodiments, provided herein is a method for treating cancer, comprising administering to a human patient in need thereof:
(a) a human or humanized anti-human PD-1 monoclonal antibody or antigen binding fragment thereof;
(b) a human or humanized anti-human ILT4 monoclonal antibody or antigen binding fragment thereof; and
(c) docetaxel.

In some embodiments, provided herein is a method for treating cancer, comprising administering to a human patient in need thereof:
(a) a human anti-human PD-1 monoclonal antibody or antigen binding fragment thereof;
(b) a human anti-human ILT4 monoclonal antibody or antigen binding fragment thereof; and
(c) docetaxel.

In other embodiments, provided herein is a method for treating cancer, comprising administering to a human patient in need thereof:
(a) a humanized anti-human PD-1 monoclonal antibody or antigen binding fragment thereof;
(b) a humanized anti-human ILT4 monoclonal antibody or antigen binding fragment thereof; and
(c) docetaxel.

In one embodiment of various methods provided herein, the anti-human PD-1 monoclonal antibody is pembrolizumab.

In another embodiment of various methods provided herein, the anti-human PD-1 monoclonal antibody is nivolumab.

In another embodiment of various methods provided herein, the anti-human PD-1 monoclonal antibody is cemiplimab.

In certain embodiments of various methods provided herein, the anti-human ILT4 monoclonal antibody or antigen binding fragment thereof comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively.

In some embodiments of various methods provided herein, the anti-human ILT4 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9.

In other embodiments of various methods provided herein, the anti-human ILT4 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

Thus, in one specific embodiment of various methods provided herein, the method for treating cancer comprises administering to a human patient in need thereof:
(a) pembrolizumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) cisplatin; and
(d) pemetrexed.

In one specific embodiment of various methods provided herein, the method for treating cancer comprises administering to a human patient in need thereof:
(a) nivolumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) cisplatin; and
(d) pemetrexed.

In one specific embodiment of various methods provided herein, the method for treating cancer comprises administering to a human patient in need thereof:
(a) cemiplimab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) cisplatin; and
(d) pemetrexed.

In one specific embodiment of various methods provided herein, the method for treating mesothelioma comprises administering to a human patient in need thereof:
(a) pembrolizumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) cisplatin; and
(d) pemetrexed.

In one specific embodiment of various methods provided herein, the method for treating mesothelioma comprises administering to a human patient in need thereof:
(a) nivolumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) cisplatin; and
(d) pemetrexed.

In one specific embodiment of various methods provided herein, the method for treating mesothelioma comprises administering to a human patient in need thereof:
(a) cemiplimab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) cisplatin; and
(d) pemetrexed.

In some embodiments, the mesothelioma is advanced mesothelioma. In other embodiments, the mesothelioma is recurrent mesothelioma.

In one specific embodiment of various methods provided herein, the method for treating breast cancer comprises administering to a human patient in need thereof:
(a) pembrolizumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In one specific embodiment of various methods provided herein, the method for treating breast cancer comprises administering to a human patient in need thereof:
(a) nivolumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In one specific embodiment of various methods provided herein, the method for treating breast cancer comprises administering to a human patient in need thereof:
(a) cemiplimab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In some embodiments, the breast cancer is TNBC.

In one specific embodiment of various methods provided herein, the method for treating ovarian cancer comprises administering to a human patient in need thereof:
(a) pembrolizumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In one specific embodiment of various methods provided herein, the method for treating ovarian cancer comprises administering to a human patient in need thereof:
(a) nivolumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In one specific embodiment of various methods provided herein, the method for treating ovarian cancer comprises administering to a human patient in need thereof:
(a) cemiplimab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In one specific embodiment of various methods provided herein, the method for treating ovarian cancer comprises administering to a human patient in need thereof:
(a) pembrolizumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) docetaxel.

In one specific embodiment of various methods provided herein, the method for treating ovarian cancer comprises administering to a human patient in need thereof:
(a) nivolumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) docetaxel.

In one specific embodiment of various methods provided herein, the method for treating ovarian cancer comprises administering to a human patient in need thereof:
(a) cemiplimab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) docetaxel.

### 6. Dosing and Administration

Further provided herein are dosing regimens and routes of administration for treating cancer (e.g., mesothelioma, ovarian cancer, or TNBC) using a combination of a PD-1 antagonist (e.g., an anti-human PD-1 monoclonal antibody or antigen binding fragment thereof), an ILT4 antagonist (e.g., an anti-human ILT4 monoclonal antibody or antigen binding fragment thereof), and one or more chemotherapeutic agents (e.g., cisplatin, pemetrexed, paclitaxel, docetaxel, etc.).

The anti-PD-1 monoclonal antibody or antigen binding fragment thereof, the anti-ILT4 monoclonal antibody or antigen binding fragment thereof, cisplatin, pemetrexed, paclitaxel, or docetaxel may be administered by continuous infusion, or by doses administered, e.g., daily, 1-7 times per week, weekly, bi-weekly, tri-weekly, every four weeks, every five weeks, every 6 weeks, monthly, bimonthly, quarterly, semiannually, annually, etc. Doses may be administered, e.g., intravenously, subcutaneously, topically, orally, nasally, rectally, intramuscular, intracerebrally, intraspinally, or by inhalation. In certain embodiments, the doses are administered intravenously. In certain embodiments, the doses are administered subcutaneously. A total dose for a treatment interval is generally at least 0.05 μg/kg body weight, more generally at least 0.2 μg/kg, 0.5 μg/kg, 1 μg/kg, 10 μg/kg, 100 μg/kg. 0.25 mg/kg, 1.0 mg/kg. 2.0 mg/kg, 5.0 mg/ml, 10 mg/kg, 25 mg/kg, 50 mg/kg or more. Doses may also be provided to achieve a pre-determined target concentration of the antibody (e.g., anti-PD-1 antibody) or antigen binding fragment thereof in the subject's serum, such as 0.1, 0.3, 1, 3, 10, 30, 100, 300 μg/mL or more.

In some embodiments, the anti-PD-1 monoclonal antibody or antigen binding fragment thereof is administered subcutaneously or intravenously, on a weekly, biweekly, triweekly, every 4 weeks, every 5 weeks, every 6 weeks, monthly, bimonthly, or quarterly basis at 10, 20, 50, 80, 100, 200, 300, 400, 500, 1000 or 2500 mg/subject. In some specific methods, the dose of the anti-PD-1 monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of the anti-PD-1 monoclonal antibody or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, from about 200 mg to about 240 mg. In some embodiments, the dose of the anti-PD-1 monoclonal antibody or antigen binding fragment thereof is 50 mg, 75 mg, 100 mg, 125 mg, 150 mg, 175 mg, 200 mg, 225 mg, 240 mg, 250 mg, 300 mg, 400 mg, or 500 mg.

In some embodiments, the anti-ILT4 monoclonal antibody or antigen binding fragment thereof is administered subcutaneously or intravenously, on a weekly, biweekly, triweekly, every 4 weeks, every 5 weeks, every 6 weeks, monthly, bimonthly, or quarterly basis at 10, 20, 50, 80, 100, 200, 300, 400, 500, 800, 1000, 1600, 2000, or 2500 mg/subject. In some specific methods, the dose of the anti-ILT4 monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of the anti-ILT4 monoclonal antibody or antigen binding fragment thereof is from about 10 mg to about 2500 mg, from about 25 mg to about 2500 mg, from about 50 mg to about 2500 mg, from about 100 mg to about 2500 mg, from about 200 mg to about 2500 mg, from about 300 mg to about 2000 mg, from about 100 mg to about 1600 mg, from about 200 mg to about 1000 mg, from about 300 mg to about 1600 mg, from about 300 mg to about 800 mg, from about 400 mg to about 800 mg. In some embodiments, the dose of the anti-ILT4 monoclonal antibody or antigen binding fragment thereof is 50 mg, 75 mg, 100 mg, 150 mg, 200 mg, 300 mg, 400 mg, 500 mg, 600 mg, 800 mg, 1000 mg, 1600 mg, or 2000 mg.

In some embodiments, cisplatin is administered subcutaneously or intravenously, on a weekly, biweekly, triweekly, every 4 weeks, every 5 weeks, every 6 weeks, monthly, bimonthly, or quarterly basis. In some specific methods, the dose of cisplatin is from about 20 mg/m² to about 100 mg/m², from 20 mg/m² to about 90 mg/m², from 20 mg/m² to about 80 mg/m², from 30 mg/m² to about 100 mg/m², or from 35 mg/m² to about 100 mg/m². In one embodiment, the dose of cisplatin is 20 mg/m², 50 mg/m², 75 mg/m², or 100 mg/m². In one embodiment, the dose of cisplatin is 20 mg/m². In one embodiment, the dose of cisplatin is 50 mg/m². In one embodiment, the dose of cisplatin is 75 mg/m². In one embodiment, the dose of cisplatin is 100 mg/m².

In some embodiments, pemetrexed is administered subcutaneously or intravenously, on a weekly, biweekly, triweekly, every 4 weeks, every 5 weeks, every 6 weeks, monthly, bimonthly, or quarterly basis. In some specific methods, the dose of pemetrexed is from about 100 mg/m² to about 750 mg/m², from 200 mg/m² to about 700 mg/m², from 250 mg/m² to about 600 mg/m², from 300 mg/m² to about 500 mg/m², or from 350 mg/m² to about 400 mg/m². In one embodiment, the dose of pemetrexed is 200 mg/m², 250 mg/m², 375 mg/m², or 500 mg/m². In one embodiment, the dose of pemetrexed is 200 mg/m². In one embodiment, the dose of pemetrexed is 250 mg/m². In one embodiment, the dose of pemetrexed is 375 mg/m². In one embodiment, the dose of pemetrexed is 500 mg/m².

In some embodiments, paclitaxel is administered subcutaneously or intravenously, on a weekly, biweekly, triweekly, every 4 weeks, every 5 weeks, every 6 weeks, monthly, bimonthly, or quarterly basis. In some specific methods, the dose of paclitaxel is from about 10 mg/m² to about 200 mg/m², from 20 mg/m² to about 200 mg/m², from 25 mg/m² to about 125 mg/m², from 50 mg/m² to about 200 mg/m², or from 50 mg/m² to about 100 mg/m². In one embodiment, the dose of paclitaxel is 50 mg/m², 80 mg/m², 90 mg/m², or 100 mg/m². In one embodiment, the dose of paclitaxel is 50 mg/m². In one embodiment, the dose of paclitaxel is 80 mg/m². In one embodiment, the dose of paclitaxel is 90 mg/m². In one embodiment, the dose of paclitaxel is 100 mg/m².

In some embodiments, docetaxel is administered subcutaneously or intravenously, on a weekly, biweekly, triweekly, every 4 weeks, every 5 weeks, every 6 weeks, monthly, bimonthly, or quarterly basis. In some specific methods, the dose of docetaxel is from about 10 mg/m² to about 200 mg/m², from 20 mg/m² to about 200 mg/m², from 25 mg/m² to about 125 mg/m², from 50 mg/m² to about 200 mg/m², or from 50 mg/m² to about 100 mg/m². In one embodiment, the dose of docetaxel is 20 mg/m², 25 mg/m², 75 mg/m², or 100 mg/m². In one embodiment, the dose of docetaxel is 20 mg/m². In one embodiment, the dose of docetaxel is 25 mg/m². In one embodiment, the dose of docetaxel is 75 mg/m². In one embodiment, the dose of docetaxel is 100 mg/m².

In some embodiments of various methods described herein, the anti-human PD-1 monoclonal antibody or antigen binding fragment thereof is pembrolizumab, the human patient is administered 200 mg, 240 mg, or 2 mg/kg pembrolizumab, and pembrolizumab is administered once every three weeks. In one embodiment, the human patient is administered 200 mg pembrolizumab once every three weeks. In one embodiment, the human patient is administered 240 mg pembrolizumab once every three weeks. In one embodiment, the human patient is administered 2 mg/kg pembrolizumab once every three weeks.

In certain embodiments of various methods described herein, the anti-human PD-1 monoclonal antibody or antigen binding fragment thereof is pembrolizumab, the human patient is administered 400 mg pembrolizumab, and pembrolizumab is administered once every six weeks.

In other embodiments of various methods described herein, the anti-human PD-1 monoclonal antibody or antigen binding fragment thereof is nivolumab, the human patient is administered 240 mg or 3 mg/kg nivolumab, and nivolumab is administered once every two weeks. In one specific embodiment, the human patient is administered 240 mg nivolumab once every two weeks. In one specific embodiment, the human patient is administered 3 mg/kg nivolumab once every two weeks. In other embodiments of various methods described herein, the anti-human PD-1 monoclonal antibody or antigen binding fragment thereof is nivolumab, the human patient is administered 480 mg nivolumab, and nivolumab is administered once every four weeks.

In yet other embodiments of various methods described herein, the anti-human PD-1 monoclonal antibody or antigen binding fragment thereof is cemiplimab, the human patient is administered 350 mg cemiplimab, and cemiplimab is administered once every three weeks.

In still other embodiments of various methods described herein, the anti-human ILT4 monoclonal antibody or antigen binding fragment thereof comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively, the human patient is administered from about 100 to about 1600 mg anti-human ILT4 antibody, and anti-human ILT4 antibody is administered once every three weeks. In still other embodiments of various methods described herein, the anti-human ILT4 monoclonal antibody or antigen binding fragment thereof comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively, the human patient is administered 100, 200, 300, 400, 800, or 1600 mg anti-human ILT4 antibody, and anti-human ILT4 antibody is administered once every three weeks. In one specific embodiment, the human patient is administered 100 mg anti-human ILT4 antibody once every three weeks. In one specific embodiment, the human patient is administered 200 mg anti-human ILT4 antibody once every three weeks. In one specific embodiment, the human patient is administered 300 mg anti-human ILT4 antibody once every three weeks. In one specific embodiment, the human patient is administered 400 mg anti-human ILT4 antibody once every three weeks. In one specific embodiment, the human patient is administered 800 mg anti-human ILT4 antibody once every three weeks. In one specific embodiment, the human patient is administered 1600 mg anti-human ILT4 antibody once every three weeks.

In certain embodiments of various methods described herein, the human patient is administered cisplatin at a dosage of from about 20 mg/m² to about 100 mg/m² and pemetrexed at a dosage of from about 200 mg/m² to about 750 mg/m², and cisplatin and pemetrexed are administered once every three weeks.

In some embodiments of various methods described herein, the human patient is administered cisplatin at a dosage of 20 mg/m², 50 mg/m², 75 mg/m², or 100 mg/m² and pemetrexed at a dosage of 250 mg/m², 375 mg/m², or 500 mg/m², and cisplatin and pemetrexed are administered once every three weeks.

In other embodiments of various methods described herein, the human patient is administered 75 mg/m² cisplatin and 500 mg/m² pemetrexed, and cisplatin and pemetrexed are administered once every three weeks.

In certain embodiments of various methods described herein, the human patient is administered paclitaxel at a dosage of from about 50 mg/m² to about 200 mg/m² on Day 1, 8, and 15 of each three-week or four-week cycle.

In some embodiments of various methods described herein, the human patient is administered paclitaxel at a dosage of 50 mg/m², 80 mg/m², 90 mg/m², or 100 mg/m² on Day 1, 8, and 15 of each three-week or four-week cycle.

In other embodiments of various methods described herein, the human patient is administered paclitaxel at a dosage of 80 mg/m² on Day 1, 8, and 15 of each three-week cycle.

In yet other embodiments of various methods described herein, the human patient is administered paclitaxel at a dosage of 90 mg/m² on Day 1, 8, and 15 of each four-week cycle.

In certain embodiments of various methods described herein, the human patient is administered docetaxel at a dosage of from about 20 mg/m² to about 200 mg/m² every three weeks.

In some embodiments of various methods described herein, the human patient is administered docetaxel at a dosage of 50 mg/m², 75 mg/m², 90 mg/m², or 100 mg/m² every three weeks.

In other embodiments of various methods described herein, the human patient is administered docetaxel at a dosage of 75 mg/m² every three weeks.

In yet other embodiments of various methods described herein, the three-week cycle dosage of docetaxel can be divided by three and administered every week. In one embodiment, the human patient is administered docetaxel at a dosage of 25 mg/m² every week. Thus, in some embodiments of various methods provided herein, the human patient is administered:
(a) 200 mg, 240 mg, or 2 mg/kg pembrolizumab;
(b) 100, 200, 300, 400, 800, 1000, or 1600 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² cisplatin; and
(d) 500 mg/m² pemetrexed;
wherein each of (a)-(d) is administered once every three weeks.

In certain embodiments of various methods provided herein, the human patient is administered:
(a) 200 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² cisplatin; and
(d) 500 mg/m² pemetrexed;
wherein each of (a)-(d) is administered once every three weeks.

In certain embodiments of various methods provided herein, the human patient is administered:
(a) 240 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² cisplatin; and
(d) 500 mg/m² pemetrexed;
wherein each of (a)-(d) is administered once every three weeks.

In certain embodiments of various methods provided herein, the human patient is administered:
(a) 2 mg/kg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² cisplatin; and
(d) 500 mg/m² pemetrexed;
wherein each of (a)-(d) is administered once every three weeks.

In certain embodiments of various methods provided herein, the human patient is administered:
(a) 400 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² cisplatin; and
(d) 500 mg/m² pemetrexed;
wherein (a) is administered once every six weeks, and each of (b)-(d) is administered once every three weeks.

In some embodiments of various methods provided herein, the human patient is administered:
(a) 200 mg, 240 mg, or 2 mg/kg pembrolizumab;
(b) 100, 200, 300, 400, 800, 1000, or 1600 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 80 mg/m² paclitaxel;
wherein each of (a)-(b) is administered once every three weeks, and (c) is administered on Day 1, 8, and 15 of each three-week cycle.

In some embodiments of various methods provided herein, the human patient is administered:
(a) 200 mg, 240 mg, or 2 mg/kg pembrolizumab;
(b) 100, 200, 300, 400, 800, 1000, or 1600 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 90 mg/m² paclitaxel;
wherein each of (a)-(b) is administered once every three weeks, and (c) is administered on Day 1, 8, and 15 of each four-week cycle.

In some embodiments of various methods provided herein, the human patient is administered:
(a) 200 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 80 mg/m² paclitaxel;
wherein each of (a)-(b) is administered once every three weeks, and (c) is administered on Day 1, 8, and 15 of each three-week cycle.

In some embodiments of various methods provided herein, the human patient is administered:
(a) 200 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 90 mg/m² paclitaxel;
wherein each of (a)-(b) is administered once every three weeks, and (c) is administered on Day 1, 8, and 15 of each four-week cycle.

In some embodiments of various methods provided herein, the human patient is administered:
(a) 400 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 80 mg/m² paclitaxel;
wherein (a) is administered once every six weeks, (b) is administered once every three weeks, and (c) is administered on Day 1, 8, and 15 of each three-week cycle.

In some embodiments of various methods provided herein, the human patient is administered:
(a) 400 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 90 mg/m² paclitaxel;
wherein (a) is administered once every six weeks, (b) is administered once every three weeks, and (c) is administered on Day 1, 8, and 15 of each four-week cycle.

In some embodiments of various methods provided herein, the human patient is administered:
(a) 200 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² docetaxel;
wherein each of (a)-(c) is administered once every three weeks.

In some embodiments of various methods provided herein, the human patient is administered:
(a) 200 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 25 mg/m² docetaxel;
wherein each of (a)-(b) is administered once every three weeks, and (c) is administered once every week.

In some embodiments of various methods provided herein, the human patient is administered:
(a) 400 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 75 mg/m² docetaxel;
wherein (a) is administered once every six weeks, and each of (b)-(c) is administered once every three weeks.

In some embodiments of various methods provided herein, the human patient is administered:
(a) 400 mg pembrolizumab;
(b) 800 mg an anti-human ILT4 antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) 25 mg/m² docetaxel;
wherein (a) is administered once every six weeks, (b) is administered once every three weeks, and (c) is administered once every week.

In some embodiments, at least one of the therapeutic agents (e.g., the anti-PD-1 monoclonal antibody or binding fragment thereof, the anti-ILT4 monoclonal antibody or binding fragment thereof, cisplatin, pemetrexed, paclitaxel, or docetaxel) in the combination therapy is administered using the same dosage regimen (dose, frequency, and duration of treatment) that is typically employed when the agent is used as monotherapy for treating the same condition. In other embodiments, the patient receives a lower total amount of at least one of the therapeutic agents (e.g., the anti-PD-1 monoclonal antibody or binding fragment thereof, the anti-ILT4 monoclonal antibody or binding fragment thereof, cisplatin, pemetrexed, paclitaxel, or docetaxel) in the combination therapy than when the agent is used as monotherapy, e.g., smaller doses, less frequent doses, and/or shorter treatment duration.

A combination therapy disclosed herein may be used prior to or following surgery to remove a tumor and may be used prior to, during, or after radiation treatment.

In some embodiments, a combination therapy disclosed herein is administered to a patient who has not previously been treated with a therapeutic agent, i.e., is treatment-naïve. In other embodiments, the combination therapy is administered to a patient who failed to achieve a sustained response after prior therapy with a therapeutic agent, i.e., is treatment-experienced.

The therapeutic combination disclosed herein may be used in combination with one or more other active agents, including but not limited to, other anti-cancer agents that are used in the prevention, treatment, control, amelioration, or reduction of risk of a particular disease or condition (e.g., cancer). Such other active agents may be administered, by a route and in an amount commonly used therefor, contemporaneously or sequentially with a therapeutic combination of the present disclosure.

The one or more additional active agents may be co-administered with the anti-PD-1 monoclonal antibody or antigen binding fragment thereof, the anti-ILT4 monoclonal antibody or antigen binding fragment thereof, cisplatin, pemetrexed, paclitaxel, or docetaxel. The additional active agent(s) can be administered in a single dosage form with one or more co-administered agent selected from the anti-PD-1 monoclonal antibody or antigen binding fragment thereof, the anti-ILT4 monoclonal antibody or antigen binding fragment thereof, cisplatin, pemetrexed, paclitaxel, and docetaxel. The additional active agent(s) can also be administered in separate dosage form(s) from the dosage forms containing the anti-PD-1 monoclonal antibody or antigen binding fragment thereof, the anti-ILT4 monoclonal antibody or antigen binding fragment thereof, cisplatin, pemetrexed, paclitaxel, or docetaxel.

### 7. Kits

In still another aspect, provided herein are kits comprising a PD-1 antagonist (e.g., an anti-PD-1 monoclonal antibody or antigen binding fragment thereof), an ILT4 antagonist (e.g., an anti-ILT4 monoclonal antibody or antigen binding fragment thereof), one or more chemotherapeutic agents (e.g., cisplatin, pemetrexed, paclitaxel, or docetaxel), packaged into suitable packaging material. A kit optionally includes a label or packaging insert that include a description of the components or instructions for use in vitro, in vivo, or ex vivo, of the components therein.

In some embodiments, the kit comprises
(a) an anti-human PD-1 monoclonal antibody or antigen binding fragment thereof;
(b) an anti-human ILT4 monoclonal antibody or antigen binding fragment thereof;
(c) cisplatin; and
(d) pemetrexed.

In some embodiments, the kit comprises
(a) an anti-human PD-1 monoclonal antibody or antigen binding fragment thereof;
(b) an anti-human ILT4 monoclonal antibody or antigen binding fragment thereof; and
(c) paclitaxel.

In some embodiments, the kit comprises
(a) an anti-human PD-1 monoclonal antibody or antigen binding fragment thereof;
(b) an anti-human ILT4 monoclonal antibody or antigen binding fragment thereof; and
(c) docetaxel.

In certain embodiments, the kit further comprises instructions for administering to a human patient the anti-human PD-1 monoclonal antibody or antigen binding fragment thereof, the anti-human ILT4 monoclonal antibody or antigen binding fragment thereof, cisplatin, and pemetrexed.

In some embodiments, the anti-PD-1 monoclonal antibody or antigen binding fragment thereof is pembrolizumab. In some embodiments, the anti-PD-1 monoclonal antibody or antigen binding fragment thereof is nivolumab. In some embodiments, the anti-PD-1 monoclonal antibody or antigen binding fragment thereof is cemiplimab.

In one embodiment, anti-human ILT4 monoclonal antibody or antigen binding fragment thereof comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively.

In another embodiment, the kit comprises: (a) pembrolizumab; (b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; (c) cisplatin; (d) pemetrexed; and (e) instructions for administering to a human patient the anti-human PD-1 monoclonal antibody or antigen binding fragment thereof, the anti-human ILT4 monoclonal antibody or antigen binding fragment thereof, cisplatin, and pemetrexed.

In another embodiment, the kit comprises: (a) pembrolizumab; (b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; (c) paclitaxel; and (d) instructions for administering to a human patient the anti-human PD-1 monoclonal antibody or antigen binding fragment thereof, the anti-human ILT4 monoclonal antibody or antigen binding fragment thereof, and paclitaxel.

In another embodiment, the kit comprises: (a) pembrolizumab; (b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; (c) docetaxel; and (d) instructions for administering to a human patient the anti-human PD-1 monoclonal antibody or antigen binding fragment thereof, the anti-human ILT4 monoclonal antibody or antigen binding fragment thereof, and docetaxel.

In some embodiments, the kit comprises means for separately retaining the components, such as a container, divided bottle, or divided foil packet. A kit of this disclosure can be used for administration of different dosage forms, for example, oral and parenteral, for administration of the separate compositions at different dosage intervals, or for titration of the separate compositions against one another.

### 8. Uses of a Therapeutic Combination for Treating Cancer

In still another aspect, provided herein are uses of a therapeutic combination for treating cancer (e.g., mesothelioma, ovarian cancer, or TNBC) in a human patient, wherein the therapeutic combination comprises:
(a) a PD-1 antagonist;
(b) an ILT4 antagonist; and
(c) one or more chemotherapeutic agents.

In some embodiments of various methods described herein, the one or more chemotherapeutic agents comprise cisplatin and pemetrexed. In certain embodiments, the one or more chemotherapeutic agents comprise paclitaxel. In certain embodiments, the one or more chemotherapeutic agents comprise docetaxel.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., NSCLC), pleural mesothelioma, gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In some embodiments, the cancer is relapsed. In other embodiments, the cancer is refractory. In yet other embodiments, the cancer is relapsed and refractory.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is NSCLC. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelfibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer.

In one embodiment, the cancer is advanced mesothelioma. In another embodiment, the cancer is recurrent mesothelioma. In yet another embodiment, the cancer is TNBC.

In one embodiment, provided herein is use of a therapeutic combination for treating mesothelioma in a human patient, wherein the therapeutic combination comprises:
(a) a PD-1 antagonist;
(b) an ILT4 antagonist;
(c) cisplatin; and
(d) pemetrexed.

In some embodiments, provided herein is use of a therapeutic combination for treating advanced mesothelioma in a human patient, wherein the therapeutic combination comprises:
(a) a PD-1 antagonist;
(b) an ILT4 antagonist;
(c) cisplatin; and
(d) pemetrexed.

In other embodiments, provided herein is use of a therapeutic combination for treating recurrent mesothelioma in a human patient, wherein the therapeutic combination comprises:
(a) a PD-1 antagonist;
(b) an ILT4 antagonist;
(c) cisplatin; and
(d) pemetrexed.

In yet other embodiments, provided herein is use of a therapeutic combination for treating breast cancer in a human patient, wherein the therapeutic combination comprises:
(a) a PD-1 antagonist;
(b) an ILT4 antagonist; and
(c) paclitaxel.

In yet other embodiments, provided herein is use of a therapeutic combination for treating TNBC in a human patient, wherein the therapeutic combination comprises:
(a) a PD-1 antagonist;
(b) an ILT4 antagonist; and
(c) paclitaxel.

In one embodiment, provided herein is use of a therapeutic combination for treating ovarian cancer in a human patient, wherein the therapeutic combination comprises:
(a) a PD-1 antagonist;
(b) an ILT4 antagonist; and
(c) paclitaxel.

In some embodiments, provided herein is use of a therapeutic combination for treating ovarian cancer in a human patient, wherein the therapeutic combination comprises:
(a) a PD-1 antagonist;
(b) an ILT4 antagonist; and
(c) docetaxel.

In still other embodiments, provided herein is use of a therapeutic combination for treating cancer, wherein the therapeutic combination comprises:
(a) an anti-human PD-1 monoclonal antibody or antigen binding fragment thereof disclosed in Section V.2;
(b) an anti-human ILT4 monoclonal antibody or antigen binding fragment thereof disclosed in Section V.3; and
(c) one or more therapeutic agents disclosed in Section V.4.

In some embodiments of various uses of a therapeutic combination for treating cancer, the one or more therapeutic agents comprise an alkylating agent. In other embodiments of various uses described herein, the one or more therapeutic agents comprise an antimetabolite. In yet other embodiments of various uses described herein, the one or more therapeutic agents comprise an alkylating agent and an antimetabolite. In one embodiment, the alkylating agent is carboplatin. In another embodiment, the antimetabolite is pemetrexed. In yet another embodiment, the one or more therapeutic agents comprise an alkylating agent and an antimetabolite, the alkylating agent is carboplatin, and the antimetabolite is pemetrexed.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

In certain embodiments, the PD-1 antagonist is an anti-human PD-L1 monoclonal antibody or antigen binding fragment thereof. In some embodiments, the anti-human PD-L1 monoclonal antibody is a human antibody. In other embodiments, the anti-human PD-L1 monoclonal antibody is a humanized antibody.

In certain embodiments, the ILT4 antagonist is an anti-human ILT4 monoclonal antibody or antigen binding fragment thereof. In some embodiments, the anti-human ILT4 monoclonal antibody is a human antibody. In other embodiments, the anti-human ILT4 monoclonal antibody is a humanized antibody.

In some embodiments of various uses provided herein, the anti-PD-1 monoclonal antibody or antigen binding fragment thereof is pembrolizumab. In some embodiments of various uses provided herein, the anti-PD-1 monoclonal antibody or antigen binding fragment thereof is nivolumab. In some embodiments of various uses provided herein, the anti-PD-1 monoclonal antibody or antigen binding fragment thereof is cemiplimab.

In certain embodiments of various uses provided herein, the anti-human ILT4 monoclonal antibody or antigen binding fragment thereof comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively.

In some embodiments of various uses provided herein, the anti-human ILT4 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9.

In other embodiments of various uses provided herein, the anti-human ILT4 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

Thus, in one specific embodiment, provided herein is use of a therapeutic combination for treating cancer, wherein the therapeutic combination comprises:
(a) pembrolizumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) cisplatin; and
(d) pemetrexed.

In one specific embodiment, provided herein is use of a therapeutic combination for treating cancer, wherein the therapeutic combination comprises:
(a) nivolumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) cisplatin; and
(d) pemetrexed.

In one specific embodiment, provided herein is use of a therapeutic combination for treating cancer, wherein the therapeutic combination comprises:
(a) cemiplimab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) cisplatin; and
(d) pemetrexed.

In one specific embodiment, provided herein is use of a therapeutic combination for treating mesothelioma, wherein the therapeutic combination comprises:
(a) pembrolizumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) cisplatin; and
(d) pemetrexed.

In one specific embodiment, provided herein is use of a therapeutic combination for treating mesothelioma, wherein the therapeutic combination comprises:
(a) nivolumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) cisplatin; and
(d) pemetrexed.

In one specific embodiment, provided herein is use of a therapeutic combination for treating mesothelioma, wherein the therapeutic combination comprises:
(a) cemiplimab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively;
(c) cisplatin; and
(d) pemetrexed.

In some embodiments, the mesothelioma is advanced mesothelioma. In other embodiments, the mesothelioma is recurrent mesothelioma.

In one specific embodiment, provided herein is use of a therapeutic combination for treating breast cancer, wherein the therapeutic combination comprises:
(a) pembrolizumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In one specific embodiment, provided herein is use of a therapeutic combination for treating breast cancer, wherein the therapeutic combination comprises:
(a) nivolumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In one specific embodiment, provided herein is use of a therapeutic combination for treating breast cancer, wherein the therapeutic combination comprises:
(a) cemiplimab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In some embodiments, the breast cancer is TNBC.

In one specific embodiment, provided herein is use of a therapeutic combination for treating ovarian cancer, wherein the therapeutic combination comprises:
(a) pembrolizumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In one specific embodiment, provided herein is use of a therapeutic combination for treating ovarian cancer, wherein the therapeutic combination comprises:
(a) nivolumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In one specific embodiment, provided herein is use of a therapeutic combination for treating ovarian cancer, wherein the therapeutic combination comprises:
(a) cemiplimab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) paclitaxel.

In one specific embodiment, provided herein is use of a therapeutic combination for treating ovarian cancer, wherein the therapeutic combination comprises:
(a) pembrolizumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) docetaxel.

In one specific embodiment, provided herein is use of a therapeutic combination for treating ovarian cancer, wherein the therapeutic combination comprises:
(a) nivolumab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) docetaxel.

In one specific embodiment, provided herein is use of a therapeutic combination for treating ovarian cancer, wherein the therapeutic combination comprises:
(a) cemiplimab;
(b) an anti-human ILT4 monoclonal antibody or antigen binding fragment thereof that comprises a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively; and
(c) docetaxel.

In various embodiments of the uses described herein, the dosing regimens of each therapeutic agent in the therapeutic combination can be any dosing regimens disclosed in this specification. The administration routes of each therapeutic agent in the therapeutic combination can be any administration routes disclosed in this specification.

A number of embodiments of the invention have been described. It will be understood that various modifications may be made without departing from the spirit and scope of the invention. It will be further understood that each embodiment may be combined with one or more other embodiments, to the extent that such a combination is consistent with the description of the embodiments.

### VI. EXAMPLES

The examples in this section (section VI) are offered by way of illustration, and not by way of limitation.

### 6.1 Example 1: Clinical Trial of Administering an Anti-PD-1 Antibody in Combination with an Anti-ILT4 Antibody, or in Combination with an Anti-ILT4 Antibody, and One or More Chemotherapeutic Agents in Solid Tumor Patients

A phase 1 open label, multi-arm, multi-center study of an anti-ILT4 antibody (“MK-4830”) as monotherapy or in combination with pembrolizumab for participants with advanced solid tumors has been ongoing (NCT03564691).

At the time of the May 10, 2019 data cutoff, a total of 51 participants had received at least 1 dose of MK-4830. In the ongoing MK-4830 monotherapy phase, MK-4830 monotherapy had reached the maximum planned dose level of 1600 mg. In the ongoing MK-4830 plus pembrolizumab combination phase, MK-4830 dose level had reached a maximum dose of 800 mg.

The target dose limiting toxicity (DLT) rate was not reached in any of the cohorts and the maximum tolerated dose (MTD) was not defined.

Of the total 51 treated participants, preliminary data showed that 26 (51%) had one or more drug-related AEs, the majority of which were Grade 1. The most common study-drug related AEs in all participants were arthralgia (9.8%), fatigue (9.8%), decreased appetite (7.8%), diarrhea (7.8%), and pruritus (7.8%). There was one episode of transient Grade 3 blood pressure increase at the 1600 mg dose level in the MK-4830 monotherapy cohort. There were no Grade 4 or 5 drug-related AEs or SAEs.

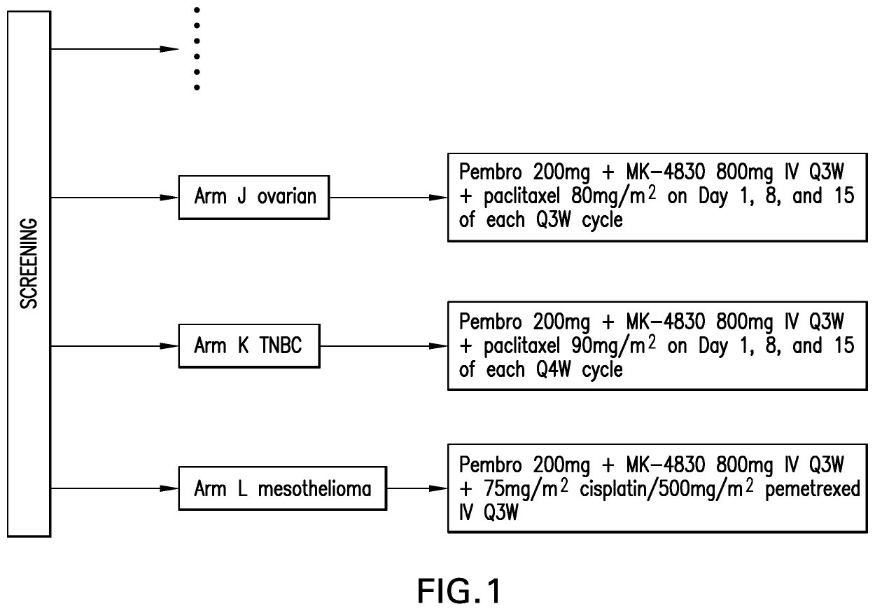

Based on the preliminary data, an expansion phase in specific indications (e.g., mesothelioma, ovarian cancer, and TNBC) is incorporated to support the study. FIG. 1 illustrate a schema of the trial design. Arm J is to test the combination of 800 mg MK-4830 Q3W, 200 mg pembrolizumab Q3W, and 80 mg/m² paclitaxel on Day 1, 8, and 15 of each Q3W cycle in ovarian cancer patients; Arm K is to test the combination of 800 mg MK-4830 Q3W, 200 mg pembrolizumab Q3W, and 90 mg/m² paclitaxel on Day 1, 8, and 15 of each Q4W cycle in TNBC patients; Arm L is to test the combination of 800 mg MK-4830 Q3W, 200 mg pembrolizumab Q3W, 75 mg/m² cisplatin and 500 mg/m² pemetrexed Q3W in mesothelioma patients.

The primary objectives in the expansion phase are to determine the safety and tolerability of MK-4830 administered in combination with pembrolizumab and paclitaxel (Arms J and K) or in combination with pembrolizumab+cisplatin/pemetrexed (Arm L); and to evaluate the objective response rate (ORR) based on Response Evaluation Criteria In Solid Tumors version 1.1 (RECIST 1.1 (mRECIST for Arm L)) assessed by the investigator in participants treated with MK-4830 in combination with pembrolizumab and chemotherapeutic agents (Arms J, K, and L).

The secondary objective in the expansion phase is to evaluate the pharmacokinetics (PK) of MK-4830 administered in combination with pembrolizumab and chemotherapeutic agents (Arms J, K, and L).

The tertiary/exploratory objectives in the expansion phase include to evaluate the development of circulating anti-MK-4830 antibodies and anti-pembrolizumab antibodies following the administration of MK-4830 in combination with pembrolizumab and chemotherapeutic agents (Arms J, K, and L); to evaluate the PK of pembrolizumab administered in combination with MK-4830 and chemotherapeutic agents (Arms J, K, and L).

The primary outcome measures include:
1. Dose-Limiting Toxicities (DLTs) [Time Frame: Cycle 1 (Up to 21 days)] The occurrence of the following toxicities, if assessed by the investigator to be possibly, probably, or definitely related to study treatment administration:
   Grade 4 nonhematologic toxicity (not laboratory)
   Grade 4 hematologic toxicity lasting >=7 days, except thrombocytopenia:
   Any nonhematologic AE >=Grade 3 in severity, with exceptions
   Any Grade 3 or Grade 4 alanine aminotransferase, aspartate aminotransferase, and/or bilirubin laboratory value
   Any other nonhematologic laboratory value if: Clinically significant medical intervention is required to treat the participant, OR The abnormality leads to hospitalization, OR The abnormality persists for >1 week, OR The abnormality results in a drug-induced liver injury
   Febrile neutropenia Grade 3 or Grade 4
   Prolonged delay (>2 weeks) in initiating Cycle 2 due to treatment-related toxicity.
   Any treatment-related toxicity that causes the participant to discontinue treatment during Cycle 1.
   Grade 5 toxicity
2. Adverse Events (AEs) [Time Frame: Up to approximately 27 months] Number of participants who experienced an AE. An AE is defined as any unfavorable and unintended sign including an abnormal laboratory finding, symptom or disease associated with the use of a medical treatment or procedure, regardless of whether it is considered related to the medical treatment or procedure, that occurs during the course of the study.
3. Study Treatment Discontinuations Due to AEs [Time Frame: Up to approximately 24 months]

Number of participants who discontinue from treatment due to an AE. The secondary outcome measures include:
1. Area Under the Curve (AUC) of Plasma MK-4830 [Time Frame: 24 hours pre-infusion and end of infusion on Day 1 of Cycles 1 to 4, 6, and 8, and every 4 cycles thereafter and 2 hours post-infusion on Day 1 of Cycles 1 to 4, 6, and 8 and on Days 8 and Day 15 in Cycles 1 to 3 (Up to approximately 24 months)]
2. Minimum Drug Concentration (Cmin) of Plasma MK-4830 [Time Frame: 24 hours pre-infusion and end of infusion on Day 1 of Cycles 1 to 4, 6, and 8, and every 4 cycles thereafter and 2 hours post-infusion on Day 1 of Cycles 1 to 4, 6, and 8 and on Days 8 and Day 15 in Cycles 1 to 3 (Up to approximately 24 months)]
3. Maximum Drug Concentration (Cmax) of Plasma MK-4830 [Time Frame: 24 hours pre-infusion and end of infusion on Day 1 of Cycles 1 to 4, 6, and 8, and every 4 cycles thereafter and 2 hours post-infusion on Day 1 of Cycles 1 to 4, 6, and 8 and on Days 8 and Day 15 in Cycles 1 to 3 (Up to approximately 24 months)]

The tertiary/exploratory outcome measures include:
1. Circulating anti-MK-4830 antibody level
2. Circulating anti-pembrolizumab antibody level
3. AUC of Plasma pembrolizumab [Time Frame: 24 hours pre-infusion and end of infusion on Day 1 of Cycles 1 to 4, 6, and 8, and every 4 cycles thereafter and 2 hours post-infusion on Day 1 of Cycles 1 to 4, 6, and 8 and on Days 8 and Day 15 in Cycles 1 to 3 (Up to approximately 24 months)]
4. Cmin of Plasma pembrolizumab [Time Frame: 24 hours pre-infusion and end of infusion on Day 1 of Cycles 1 to 4, 6, and 8, and every 4 cycles thereafter and 2 hours post-infusion on Day 1 of Cycles 1 to 4, 6, and 8 and on Days 8 and Day 15 in Cycles 1 to 3 (Up to approximately 24 months)]
5. Cmax of Plasma pembrolizumab [Time Frame: 24 hours pre-infusion and end of infusion on Day 1 of Cycles 1 to 4, 6, and 8, and every 4 cycles thereafter and 2 hours post-infusion on Day 1 of Cycles 1 to 4, 6, and 8 and on Days 8 and Day 15 in Cycles 1 to 3 (Up to approximately 24 months)]

Male and female participants at least 18 years of age with advanced ovarian cancer, TNBC, or mesothelioma are enrolled in this study.

Inclusion criteria for Arm J (ovarian cancer):
Must have histologically confirmed high-grade epithelial (including serous or predominantly serous, endometrioid, non-mucinous, non-borderline, or clear cell) ovarian, fallopian tube or primary peritoneal carcinoma.
Has received 1 or 2 prior lines of systemic therapy, including at least 1 prior platinum-based therapy.
Note: prior PARP inhibitors and prior bevacizumab are permitted. Prior PD-1/PD-L1 use is permitted if treatment was not discontinued due to toxicity.
Has radiographic evidence of disease progression by RECIST within 6 months after the last dose of platinum-based chemotherapy for ovarian cancer (i.e., platinum-resistant disease).
Note: participants with platinum-refractory ovarian cancer (defined as disease that has progressed while receiving platinum-based therapy) are not eligible.
Is a candidate for paclitaxel chemotherapy.
Note: If patient cannot tolerate paclitaxel, docetaxel may be considered after Sponsor consultation.

Inclusion criteria for Arm K (TNBC):
Have locally recurrent inoperable breast cancer not previously treated with chemotherapy and which cannot be treated with curative intent, or have metastatic breast cancer not previously treated with chemotherapy.
Have centrally confirmed TNBC, as defined by the most recent ASCO/CAP guidelines.
Note: Subjects initially diagnosed with hormone receptor-positive and/or HER2-positive breast cancer must have central confirmation of TNBC in a tumor biopsy obtained from a local recurrence or distant metastasis site.
Have completed treatment for Stage I-III breast cancer, if indicated, and ≥6 months elapsed between the completion of treatment with curative intent (e.g., date of primary breast tumor surgery or date of last adjuvant chemotherapy administration, whichever occurred last) and first documented local or distant disease recurrence.
Note: Adjuvant radiation therapy is not considered treatment with curative intent for the purpose of calculating the ≥6 month interval requirement described above.
Note: First documentation of local or distant disease recurrence must be in the form of a dated biopsy, pathology, or imaging study report. A laboratory report indicating tumor marker elevation cannot be used as documentation of local or distant disease recurrence, unless accompanied by dated biopsy, pathology, or imaging study report.
Note: If the participant received a taxane in a prior neo (adjuvant) setting, then ≥12 months must have elapsed between the completion of treatment with curative intent (e.g., date of primary breast tumor surgery or date of last adjuvant chemotherapy administration, whichever occurred last) and first documented local or distant disease recurrence.
Have been treated with (neo) adjuvant anthracycline, if they received systemic treatment in the (neo) adjuvant setting, unless anthracycline was contraindicated or not considered the best treatment option for the participant in the opinion of the treating physician.
Note: Participants presenting with de novo metastatic TNBC are eligible for the study, if anthracycline is contraindicated or not considered the best treatment option for the subject in the opinion of the treating physician.

Inclusion criteria for Arm L (advanced mesothelioma):
Have histologically confirmed diagnosis of recurrent and/or advanced mesothelioma that is considered incurable by standard therapies.
Be eligible to receive standard chemotherapy with pemetrexed and cisplatin (or carboplatin) and have no contraindications to standard chemotherapy.

Exclusion criteria for Arm J (ovarian cancer):
Has non-epithelial cancers, including borderline (low malignant potential), malignant Müllerian mixed tumor (carcinosarcoma), mucinous, seromucinous predominantly mucinous, malignant Brenner's tumor and undifferentiated carcinoma and/or germ cell tumors and/or sex cord-stromal tumors.
Has received more than 2 prior lines of systemic therapy for ovarian cancer.

Exclusion criteria for Arm K (TNBC):
Has a known history of hypersensitivity or allergy to the study chemotherapies (paclitaxel) and/or any of their components.
Is receiving any medication prohibited in combination with study chemotherapies as described in the respective product labels, unless medication was stopped within 7 days prior to randomization.

Exclusion criteria for Arm L (advanced mesothelioma):
Has a known history of hypersensitivity or allergy the study chemotherapies (e.g., pemetrexed, cisplatin) and/or any of their components.
Is receiving any medication prohibited in combination with study chemotherapies as described in the respective product labels, unless medication was stopped within 7 days prior to randomization.

The study treatments are outlined below in Table 3.

TABLE 1
Study Treatments

| Study | | Unit Dose | | | | | Regimen/ |
|---|---|---|---|---|---|---|---|
| Treatment | Dosage | Strength(s)/ | Dose | Dosage | | Route of | Treatment |
| Name | Formulation | Potency | Frequency | Level(s) | | Administration | Period |
| MK-4830 | Solution for | 100 mg/vial | Every 3 weeks | 800 | mg | IV infusion | Day 1 of each 21- |
| All Arms | infusion | | (Q3W) | | | | day cycle for up to |
| | | | | | | | 35 cycles |
| Pembrolizumab | Solution for | 100 mg/vial | Every 3 weeks | 200 | mg | IV infusion | Day 1 of each 21- |
| All Arms | infusion | | (Q3W) | | | | day cycle for up to |
| | | | | | | | 35 cycles |
| Paclitaxel | Solution for | Variable | Every 1 week | 80 | mg/m² | IV infusion | Days 1, 8 and 15 |
| Arm J | infusion | | (QW) | | | | of each 21-day |
| (Ovarian) | | | | | | | cycle until disease |
| | | | | | | | progression or |
| | | | | | | | prohibitive |
| | | | | | | | toxicity |
| Docetaxel: | Solution for | Variable | Every 3 weeks | 75 mg/m² | | IV infusion | Q3W or QW until |
| Arm J only | infusion | | or every 1 | (Q3W) or | | | disease |
| (Ovarian) | | | week | 25 mg/m² | | | progression or |
| | | | (Q3W or QW) | (QW) | | | prohibitive |
| | | | | | | | toxicity |
| Paclitaxel: | Solution for | Variable | Every 4 weeks | 90 | mg/m² | IV infusion | Days 1, 8 and 15 |
| Arm K | infusion | | (Q4W) | | | | Q4W until disease |
| (TNBC) | | | | | | | progression or |
| | | | | | | | prohibitive |
| | | | | | | | toxicity |
| Cisplatin | Solution for | Variable | Every 3 weeks | 75 | mg/m² | IV infusion | Day 1 of each 21- |
| Arm L | infusion | | (Q3W) | | | | day cycle for |
| (Mesothelioma) | | | | | | | maximum 6 cycles |
| Pemetrexed | Solution for | 500 mg/m² | Every 3 weeks | 500 | mg/m² | IV infusion | Day 1 of each 21- |

| | | | | |
|---|---|---|---|---|
| Arm L | infusion | (with vitamin | (Q3W) | day cycle for |
| (Mesothelioma) | | supplement) | | maximum 6 cycles |

Docetaxel (75 mg/m² Q3W or 25 mg/m² QW) may be considered for ovarian cancer participants who experience either a severe hypersensitivity reaction to paclitaxel or an adverse event requiring discontinuation of paclitaxel only after consultation with the Sponsor.

MK-4830 is administered at the dose specified in the specific arm of the study as an IV infusion every 3 weeks (Q3W). MK-4830 is administered after completion of pembrolizumab infusion. Pembrolizumab and MK-4830 are administered prior to chemotherapeutic agents.

### VII. SEQUENCE LISTING

Table 4 below summarizes all sequences disclosed in the present specification.

TABLE 4
SEQ ID NOS and Corresponding Sequences

| SEQ | De- | |
|---|---|---|
| ID | scrip- | |
| NO | tion | Sequence |
| 1 | MK- | TGSSSNIGAGYDVH |
| | 4830, | |
| | VL- | |
| | CDR1 | |
| 2 | MK- | GDSNRPS |
| | 4830, | |
| | VL- | |
| | CDR2 | |
| 3 | MK- | QSFDNSLSAYV |
| | 4830, | |
| | VL- | |
| | CDR3 | |
| 4 | MK- | ESVLTQPPSVSGAPGQRVTISCTGSSSNIGAGYDVHWYQ |
| | 4830, | QLPGTAPKLLIYGDSNRPSGVPDRFSVSKSGASASLAIT |
| | VL | GLQAEDEADYYCQSFDNSLSAYVFGGGTQLTVL |
| 5 | MK- | ESVLTQPPSVSGAPGQRVTISCTGSSSNIGAGYDVHWYQ |
| | 4830, | QLPGTAPKLLIYGDSNRPSGVPDRFSVSKSGASASLAIT |
| | light | GLQAEDEADYYCQSFDNSLSAYVFGGGTQLTVLGQPKA |
| | chain | APSVTLFPPSSEELQANKATLVCLISDFYPGAVTVAWKA |
| | | DSSPVKAGVETTTPSKQSNNKYAASSYLSLTPEQWKSH |
| | | RSYSCQVTHEGSTVEKTVAPTECS |
| 6 | MK- | GYYWS |
| | 4830, | |
| | VH- | |
| | CDR1 | |
| 7 | MK- | EINHAGSTNYNPSLKS |
| | 4830, | |
| | VH- | |
| | CDR2 | |
| 8 | MK- | LPTRWVTTRYFDL |
| | 4830, | |
| | VH- | |
| | CDR3 | |
| 9 | MK- | EVQLQQWGAGLLKPSETLSLTCAVYGGSFSGYYWSWI |
| | 4830, | RQPPGKGLEWIGEINHAGSTNYNPSLKSRVTISVDTSKN |
| | VH | QFSLKLSSVTAADTAVYYCARLPTRWVTTRYFDLWGR |
| | | GTLVTVSS |
| 10 | MK- | EVQLQQWGAGLLKPSETLSLTCAVYGGSFSGYYWSWI |
| | 4830, | RQPPGKGLEWIGEINHAGSTNYNPSLKSRVTISVDTSKN |
| | heavy | QFSLKLSSVTAADTAVYYCARLPTRWVTTRYFDLWGR |
| | chain | GTLVTVSSASTKGPSVFPLAPCSRSTSESTAALGCLVKD |
| | | YFPEPVTVSWNSGALTSGVHTFPAVLQSSGLYSLSSVVT |
| | | VPSSSLGTKTYTCNVDHKPSNTKVDKRVESKYGPPCPP |
| | | CPAPEFLGGPSVFLFPPKPKDTLMISRTPEVTCVVVDVS |
| | | QEDPEVQFNWYVDGVEVHNAKTKPREEQFNSTYRVVS |
| | | VLTVLHQDWLNGKEYKCKVSNKGLPSSIEKTISKAKGQ |
| | | PREPQVYTLPPSQEEMTKNQVSLTCLVKGFYPSDIAVE |
| | | WESNGQPENNYKTTPPVLDSDGSFFLYSRLTVDKSRWQ |
| | | EGNVFSCSVMHEALHNHYTQKSLSLSLGK |

1. A method of treating cancer, comprising administering to a human patient in need thereof:
(a) a PD-1 antagonist;
(b) an ILT4 antagonist;
(c) cisplatin; and
(d) pemetrexed.

2. The method of claim 1, wherein the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer (NSCLC), pleural mesothelioma, gastric cancer, colorectal cancer, cervical cancer, synovial sarcoma, head and neck cancer, squamous cell carcinoma, lymphoma, diffuse large B-cell lymphoma, non-Hodgkin lymphoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, and carcinoid cancer.

3-5. (canceled)

6. A kit comprising:
(a) a PD-1 antagonist;
(b) an ILT4 antagonist;
(c) cisplatin; and
(d) pemetrexed.

7-12. (canceled)

13. A method of treating cancer, comprising administering to a human patient in need thereof:
(a) a PD-1 antagonist;
(b) an ILT4 antagonist; and
(c) a mitosis inhibitor.

14. The method of claim 13, wherein the mitosis inhibitor is paclitaxel or docetaxel.

15-31. (canceled)

32. The method of claim 1, wherein the PD-1 antagonist is an anti-human PD-1 monoclonal antibody or antigen binding fragment thereof.

33. The method of claim 1, wherein the PD-1 antagonist is an anti-human PD-L1 monoclonal antibody or antigen binding fragment thereof.

34. The method of claim 32, wherein the anti-human PD-1 monoclonal antibody or antigen binding fragment thereof is a humanized antibody or a human antibody.

35. (canceled)

36. The method of claim 1, wherein the ILT4 antagonist is an anti-human ILT4 monoclonal antibody or antigen binding fragment thereof.

37. The method of claim 36, wherein the anti-human ILT4 monoclonal antibody or antigen binding fragment thereof is a humanized antibody or a human antibody.

38. (canceled)

39. The method of claim 32, wherein the anti-human PD-1 monoclonal antibody or antigen binding fragment thereof is pembrolizumab.

40. The method of claim 32, wherein the anti-human PD-1 monoclonal antibody or antigen binding fragment thereof is nivolumab or cemiplimab.

41. (canceled)

42. The method of claim 36, wherein the anti-human ILT4 monoclonal antibody or antigen binding fragment thereof comprises a V_{L} CDR1, a V_{L} CDR2, and a V_{L} CDR3 comprising amino acid sequences as set forth in SEQ ID NOs: 1, 2, and 3, respectively, and a V_{H} CDR1, a V_{H} CDR2, and a V_{H} CDR3 comprising amino acid sequences as set forth in SEQ ID NOs: 6, 7, and 8, respectively.

43. The method of claim 36, wherein the anti-human ILT4 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9.

44. The method of claim 36, wherein the anti-human ILT4 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

45-47. (canceled)

48. The method of claim 39, wherein the human patient is administered 200 mg or 2 mg/kg pembrolizumab once every three weeks.

49. The method of claim 39, wherein the human patient is administered 400 mg pembrolizumab once every six weeks.

50. The method of claim 40, wherein the human patient is administered 240 mg or 3 mg/kg nivolumab once every two weeks, 480 mg nivolumab once every four weeks, or 350 mg cemiplimab once every three weeks.

51-52. (canceled)

53. The method of claim 36, wherein the human patient is administered from about 100 mg to about 1600 mg anti-human ILT4 antibody or antigen binding fragment thereof once every three weeks.

54-57. (canceled)

58. The method of claim 14, wherein:
(a) the PD-1 antagonist is pembrolizumab; and
(b) the ILT4 antagonist is a monoclonal antibody or antigen binding fragment thereof comprising a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively.

59. The method of claim 14, wherein:
(a) the PD-1 antagonist is nivolumab or cemiplimab; and
(b) the ILT4 antagonist is a monoclonal antibody or antigen binding fragment thereof comprising a V_{L} CDR1, a V_{L} CDR2, and a V_{L} CDR3 comprising amino acid sequences as set forth in SEQ ID NOS: 1, 2, and 3, respectively, and a V_{H} CDR1, a V_{H} CDR2, and a V_{H} CDR3 comprising amino acid sequences as set forth in SEQ ID NOS: 6, 7, and 8, respectively.

60. (canceled)

61. The method of claim 58, wherein the human patient is administered 200 mg or 2 mg/kg pembrolizumab once every three weeks.

62. The method of claim 58, wherein the human patient is administered 400 mg pembrolizumab once every six weeks.

63. The method of claim 59, wherein the human patient is administered 240 mg or 3 mg/kg nivolumab once every two weeks, 480 mg nivolumab once every four weeks, or 350 mg cemiplimab once every three weeks.

64-65. (canceled)

66. The method of claim 58, wherein the human patient is administered from about 100 mg to about 1600 mg anti-human ILT4 antibody or antigen binding fragment thereof once every three weeks.

67-68. (canceled)

69. The method of claim 58, wherein the human patient is administered paclitaxel at from about 50 mg/m² to about 200 mg/m² on Day 1, 8, and 15 of each three-week or four-week cycle.

70-82. (canceled)
